(19) United States
(12) Patent Application Publication
(10) Pub. No.: US 2013/0252811 A1
(43) Pub. Date: Sep. 26, 2013

(54) NOVEL HERBICIDE

(71) Applicants: Jane Elisabeth Corbin, Berkshire (GB); Christopher John Mathews, Berkshire (GB); Glynn Mitchell, Berkshire (GB); Claudio Screpanti, Basel (CH)

(72) Inventors: Jane Elisabeth Corbin, Berkshire (GB); Christopher John Mathews, Berkshire (GB); Glynn Mitchell, Berkshire (GB); Claudio Screpanti, Basel (CH)

(73) Assignee: SYNGENTA LIMITED, Greensboro, NC (US)

(21) Appl. No.: 13/516,681

(22) PCT Filed: Dec. 10, 2010
(86) PCT No.: PCT/GB10/02268

§ 371 Date: May 29, 2013

(30) Foreign Application Priority Data
Dec. 17, 2009 (GB) 0922101.1
Nov. 29, 2010 (GB) 1020227.3
Dec. 2, 2010 (GB) 1020462.6

(51) Int. Cl. A01N 43/16; A01N 43/66; A01N 43/80; A01N 43/653; C07D 309/30; A01N 43/54

(52) U.S. Cl. CPC A01N 43/16; C07D 309/30; A01N 43/54; A01N 43/80; A01N 43/653; A01N 43/66
USPC 504/105; 504/136; 504/138; 504/139; 504/134; 504/103; 504/106; 504/292

(57) ABSTRACT

Herbicidal composition comprising a pyrandione herbicide and a co-herbicide The present invention relates to a herbicidal composition comprising as active ingredient a mixture of a) a herbicidally effective amount of a compound of formula (I) wherein: R¹ is methyl, ethyl, n-propyl, halogen, difluoromethoxy, trifluoromethoxy or trifluoromethyl, R² is phenyl or phenyl substituted by C₁-C₄ alkyl, C₁-C₄ haloalkyl, C₁-C₄ alkoxy, C₁-C₄ haloalkoxy or halogen, R⁴, R⁵, R⁶ and R⁷, independently of each other, are hydrogen or C₁-C₄ alkyl, Y is O, and G is hydrogen, an alkali metal, alkaline earth metal, sulfonium, or ammonium, or G is a latentiating group which is C(O)—R^{a} or C(0)-O—R^{b}; and b) a co-herbicide selected from the group consisting of fenoxasulfone, ipfencarbazone, propyrisulfuron, and N-[2-[(4,6-dimethoxy-1,3,5-triazin-2-yl)carbonyl]-6-fluorophenyl]-1,1-difluoro-N-methylmethanesulfonamide. The herbicidal composition is typically for controlling grasses and weeds in crops of useful plants, especially for controlling Echinochloa and/or Leptochloa weeds in crops of rice.

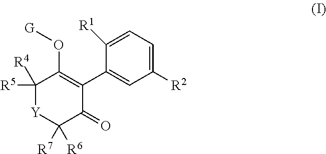

The present invention relates to new herbicidal compositions, for example for controlling grasses and weeds in crops of useful plants, especially in crops of rice; which compositions comprise a herbicidally active cyclic dione (specifically a pyrandione), a enol ketone tautomer thereof, or an enol group derivative of the enol ketone tautomer thereof; and a co-herbicide.

WO 2008/071405 A1 (Syngenta Participations AG and Syngenta Limited) discloses pyrandione, thiopyrandione and cyclohexanedione compounds, and enol ketone tautomer derivatives thereof, suitable for use as herbicides. The pyrandione and derivative compounds of formula (I), as defined hereinbelow, are generically disclosed in WO 2008/071405 A1. WO 2008/071405 A1 also discloses mixtures of these pyrandione compounds and derivatives with various mixture partners such as imazosulfuron or pyroxasulfone (KIH-485).

Pyroxasulfone and other isoxazoline herbicides were disclosed originally in EP 1 364 946 A1 (Kumiai Chemical Industry Co., Ltd and Ihara Chemical Industry Co., Ltd), derived from WO 02/062770. Pyroxasulfone an inhibitor of very long chain fatty acids (VLCFAs) and/or inhibits cell division, in plants. Pyroxasulfone (CAS Registry no. 447399-55-5) is an isoxazoline herbicide having the following structure:

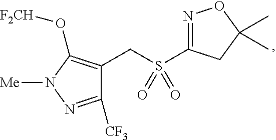

Fenoxasulfone, whose CAS registry number is 639826-16-7, has the following structure:

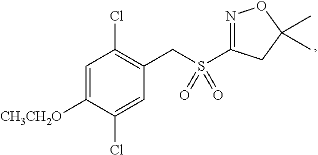

and is covered by the scope of the isoxazoline herbicides disclosed in EP 1 203 768 A1 and CA 2 380 499 A1, both derived from WO 01/012613 A1 (Kumiai Chemical Industry Co., Ltd and Ihara Chemical Industry Co., Ltd). Mixtures of isoxazoline herbicides, e.g. specifically fenoxasulfone, with various other herbicides are disclosed in JP 2004/002324 A and JP 2005/145958 A (both publications Kumiai Chemical Industry Co., Ltd and Ihara Chemical Industry Co., Ltd). EP 2 135 508 A1 (Kumiai Chemical Industry Co., Ltd), derived from WO 2008/114493, discloses a herbicidal composition containing: (A) an isoxazoline derivative, for example fenoxasulfone (which is compound no. 54 on Table 2 on page 12), and (B) a cyclohexanedione type compound, a phenylpyrazoline type compound, a sulfonyl-aminocarbonyltriazolinone type compound, or a compound selected from a list of other co-herbicides such as pinoxaden.

Imazosulfuron (CAS Registry no. 122548-33-81 is a herbicide having the following structure:

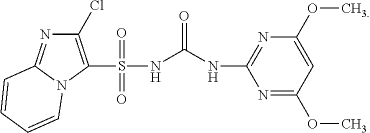 [SMILES: COc1cc(OC)nc(NC(=O)NS(=O)(=O)c2c(Cl)nc3ccccn23)n1]

The Pesticide Manual, 15^{th} edition, 2009, British Crop Production Council, entry 482 (imazosulfuron), discloses that imazosulfuron is used to control most annual (excluding Echinochloa oryzicola) and perennial broad-leaf weeds and sedges in paddy rice (at 75-95 g/ha) and turf (at 500-1000 g/ha).

It is desirable to discover new mixtures of the pyrandione compounds or enol-ketone-tautomer-derivative compounds disclosed in WO 2008/071405 A1 with other co-herbicides, especially mixtures suitable for use in crops of rice, and/or mixtures suitable to control grasses and/or weeds such as Echinochloa and/or Leptochloa which can be found in rice crops.

A first aspect of the present invention provides a herbicidal composition that comprises as active ingredient a mixture of:

a) a herbicidally effective amount of a compound of formula (I)

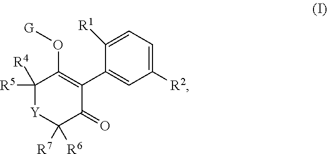

wherein

R¹ is methyl, ethyl, n-propyl, halogen, difluoromethoxy, trifluoromethoxy or trifluoromethyl,

R² is phenyl or phenyl substituted by C₁-C₄ alkyl, C₁-C₄ haloalkyl, C₁-C₄ alkoxy, C₁-C₄ haloalkoxy or halogen,

R⁴, R⁵, R⁶ and R⁷, independently of each other, are hydrogen or C₁-C₄ alkyl,

Y is O, and

G is hydrogen, an alkali metal, alkaline earth metal, sulfonium, or ammonium, or G is a latentiating group which is C(O)—R^{a} or C(O)—O—R^{b};

wherein R^{a} is C₂-C₁₈alkenyl, C₂-C₁₈alkynyl, C₁-C₁₀haloalkyl, C₁-C₁₀cyanoalkyl, C₁-C₁₀-nitroalkyl, C₁-C₁₀-aminoalkyl, C₁-C₅alkylaminoC₁-C₅alkyl, C₂-C₈dialkylaminoC₁-C₅alkyl, C₃-C₇cycloalkylC₁-C₅alkyl, C₁-C₅alkoxyC₁-C₅alkyl, C₃-C₅alkenyloxyC₁-C₅alkyl, C₃-C₅alkynyloxyC₁-C₅alkyl, C₁-C₅alkylthioC₁-C₅alkyl, C₁-C₅alkylsulfinylC₁-C₅alkyl, C₁-C₅alkylsulfonylC₁-C₅alkyl, C₂-C₈alkylideneaminoxyC₁-C₅alkyl, C₁-C₅alkylcarbonylC₁-C₅alkyl, C₁-C₅alkoxycarbonylC₁-C₅alkyl, aminocarbonylC₁-C₅alkyl, C₁-C₅alkylaminocarbonylC₁-C₅alkyl, C₂-C₈dialkylaminocarbonylC₁-C₅alkyl, C₁-C₅alkylcarbonylaminoC₁-C₅alkyl, N—C₁-C₅alkylcarbonyl-N—C₁-C₅alkylaminoC₁-C₅alkyl, C₃-C₅trialkylsilylC₁-C₅alkyl, phenylC₁-C₅alkyl (wherein the phenyl is optionally substituted by C₁-C₃alkyl, C₁-C₃haloalkyl, C₁-C₃alkoxy, C₁-C₃haloalkoxy, C₁-C₃alkylthio, C₁-C₃alkylsulfinyl, C₁-C₃alkylsulfonyl, halogen, cyano, or by nitro), heteroarylC₁-C₅alkyl (wherein the heteroaryl is optionally substituted by C₁-C₃alkyl, C₁-C₃haloalkyl, C₁-C₃alkoxy, C₁-C₃haloalkoxy, C₁-C₃alkylthio, C₁-C₃alkylsulfinyl, C₁-C₃alkylsulfonyl, halogen, cyano, or by nitro), C₂-C₅haloalkenyl, C₃-C₈cycloalkyl, phenyl or phenyl substituted by C₁-C₃alkyl, C₁-C₃haloalkyl, C₁-C₃alkoxy, C₁-C₃haloalkoxy, halogen, cyano or nitro, heteroaryl or heteroaryl substituted by C₁-C₃alkyl, C₁-C₃haloalkyl, C₁-C₃alkoxy, C₁-C₃haloalkoxy, halogen, cyano or nitro; and
R^{b} is C₁-C₁₈alkyl, C₃-C₁₈alkenyl, C₃-C₁₈alkynyl, C₂-C₁₀haloalkyl, C₁-C₁₀cyanoalkyl, C₁-C₁₀nitroalkyl, C₂-C₁₀aminoalkyl, C₁-C₅alkylaminoC₁-C₅alkyl, C₂-C₈dialkylaminoC₁-C₅alkyl, C₃-C₇cycloalkylC₁-C₅alkyl, C₁-C₅alkoxyC₁-C₅alkyl, C₃-C₅alkenyloxyC₁-C₅alkyl, C₃-C₅alkynyloxyC₁-C₅alkyl, C₁-C₅alkylthioC₁-C₅alkyl, C₁-C₅alkylsulfinylC₁-C₅alkyl, C₁-C₅alkylsulfonylC₁-C₅alkyl, C₂-C₈alkylideneaminoxyC₁-C₅alkyl, C₁-C₅alkylcarbonylC₁-C₅alkyl, C₁-C₅alkoxycarbonylC₁-C₅alkyl, aminocarbonylC₁-C₅alkyl, C₁-C₅alkylaminocarbonylC₁-C₅alkyl, C₂-C₈dialkylaminocarbonylC₁-C₅alkyl, C₁-C₅alkylcarbonylaminoC₁-C₅alkyl, N—C₁-C₅alkylcarbonyl-N—C₁-C₅alkylaminoC₁-C₅alkyl, C₃-C₆-trialkylsilylC₁-C₅alkyl, phenylC₁-C₅alkyl (wherein the phenyl is optionally substituted by C₁-C₃alkyl, C₁-C₃haloalkyl, C₁-C₃alkoxy, C₁-C₃haloalkoxy, C₁-C₃alkylthio, C₁-C₃alkylsulfinyl, C₁-C₃alkylsulfonyl, halogen, cyano, or by nitro), heteroarylC₁-C₅alkyl (wherein the heteroaryl is optionally substituted by C₁-C₃alkyl, C₁-C₃haloalkyl, C₁-C₃alkoxy, C₁-C₃haloalkoxy, C₁-C₃alkylthio, C₁-C₃alkylsulfinyl, C₁-C₃alkylsulfonyl, halogen, cyano, or by nitro), C₃-C₅haloalkenyl, C₃-C₈cycloalkyl, phenyl or phenyl substituted by C₁-C₃alkyl, C₁-C₃haloalkyl, C₁-C₃alkoxy, C₁-C₃haloalkoxy, halogen, cyano or nitro, heteroaryl or heteroaryl substituted by C₁-C₃alkyl, C₁-C₃haloalkyl, C₁-C₃alkoxy, C₁-C₃haloalkoxy, halogen, cyano or nitro;
and
b) a co-herbicide selected from the group consisting of fenoxasulfone, ipfencarbazone, propyrisulfuron, and N-[2-[(4,6-dimethoxy-1,3,5-triazin-2-yl)carbonyl]-6-fluorophenyl]-1,1-difluoro-N-methylmethanesulfonamide.

In the substituent definitions of the compounds of the formula (I), each alkyl moiety either alone or as part of a larger group such as haloalkyl or alkoxy, is a straight or branched chain having 1 to 4 carbon atoms and is, preferably, methyl, ethyl, propyl or butyl.

Preferred halogens are fluorine, chlorine and bromine.

Haloalkyl groups are alkyl groups which are substituted with one or more of the same or different halogen atoms and are, for example, CF₃, CF₂C₁, CF₂H, CCl₂H, FCH₂, ClCH₂, BrCH₂, CH₃CHF, (CH₃)₂CF, CF₃CH₂ or CHF₂CH₂.

The group G denotes hydrogen, an alkali metal cation, alkaline earth metal cation, sulfonium cation (preferably —S(C₁₋C₆alkyl₃)⁺) or ammonium cation (preferably —NH₄⁺ or −N(C₁-C₆alkyl)₄⁺), or a latentiating group. These latentiating groups G are selected to allow its removal by one or a combination of biochemical, chemical or physical processes to afford compounds of formula (I) where G is H before, during or following (preferably during or following, more preferably following) application to the treated area or plants. Examples of these processes include enzymatic cleavage, chemical hydrolysis and photoloysis. Compounds bearing such groups G may in some cases offer certain advantages, such as improved penetration of the cuticula of the plants treated, increased tolerance of crops, improved compatibility or stability in formulated mixtures containing other herbicides, herbicide safeners, plant growth regulators, fungicides or insecticides, or reduced leaching in soils.

The latentiating group G is C(O)—R⁸ or C(O)—O—R^{b}; wherein R⁸ is H, C₁-C₁₈alkyl, C₂-C₁₈alkenyl, C₂-C₁₈alkynyl, C₁-C₁₀haloalkyl, C₁-C₁₀cyanoalkyl, C₁-C₁₀nitroalkyl, C₁-C₁₀aminoalkyl, C₁-C₅alkylaminoC₁-C₅alkyl, C₂-C₈dialkylaminoC₁-C₅alkyl, C₃-C₇cycloalkylC₁-C₅alkyl, C₁-C₅alkoxyC₁-C₅alkyl, C₃-C₅alkenyloxyC₁-C₈alkyl, C₃-C₈alkynyloxyC₁-C₈alkyl, C₁-C₅alkylthioC₁-C₈alkyl, C₁-C₅alkylsulfonylC₁-C₅alkyl, C₂-C₈alkylideneaminoxyC₁-C₅alkyl, C₁-C₅alkylcarbonylC₁-C₅alkyl, C₁-C₈alkoxycarbonylC₁-C₅alkyl, aminocarbonylC₁-C₅alkyl, C₁-C₅alkylaminocarbonylC₁-C₅alkyl, C₂-C₈dialkylaminocarbonylC₁-C₅alkyl, C₁-C₅alkylcarbonylaminoC₁-C₅alkyl, N—C₁-C₅alkylcarbonyl-N—C₁-C₅alkylaminoC₁-C₅alkyl, C₃-C₈trialkylsilylC₁-C₅alkyl, phenylC₁-C₅alkyl (wherein the phenyl is optionally substituted by C₁-C₃alkyl, C₁-C₃haloalkyl, C₁-C₃alkoxy, C₁-C₃haloalkoxy, C₁-C₃alkylthio, C₁-C₃alkylsulfinyl, C₁-C₃alkylsulfonyl, halogen, cyano, or by nitro), heteroarylC₁-C₅alkyl (wherein the heteroaryl is optionally substituted by C₁-C₃alkyl, C₁-C₃haloalkyl, C₁-C₃alkoxy, C₁-C₃haloalkoxy, C₁-C₃alkylthio, C₁-C₃alkylsulfinyl, C₁-C₃alkylsulfonyl, halogen, cyano, or by nitro), C₂-C₅haloalkenyl, C₃-C₈cycloalkyl, phenyl or phenyl substituted by C₁-C₃alkyl, C₁-C₃haloalkyl, C₁-C₃alkoxy, C₁-C₃haloalkoxy, halogen, cyano or nitro, heteroaryl or heteroaryl substituted by C₁-C₃alkyl, C₁-C₃haloalkyl, C₁-C₃alkoxy, C₁-C₃haloalkoxy, halogen, cyano or nitro; and

R^{b} is C₁-C₁₈alkyl, C₃-C₁₈alkenyl, C₃-C₁₈alkynyl, C₂-C₁₀haloalkyl, C₁-C₁₀cyanoalkyl, C₁-C₁₀nitroalkyl, C₂-C₁₀aminoalkyl, C₁-C₅alkylaminoC₁-C₅alkyl, C₂-C₈dialkylaminoC₁-C₅alkyl, C₃-C₇cycloalkylC₁-C₅alkyl, C₁-C₅alkoxyC₁-C₅alkyl, C₃-C₅alkenyloxyC₁-C₅alkyl, C₃-C₅alkynyloxyC₁-C₅alkyl, C₁-C₅alkylthioC₁-C₅alkyl, C₁-C₅alkylsulfonylC₁-C₅alkyl, C₂-C₈alkylideneaminoxyC₁-C₅alkyl, C₁-C₅alkylcarbonylC₁-C₅alkyl, C₁-C₅alkoxycarbonylC₁-C₅alkyl, aminocarbonylC₁-C₅alkyl, C₁-C₅alkylaminocarbonylC₁-C₅alkyl, C₂-C₈dialkylaminocarbonylC₁-C₅alkyl, C₁-C₅alkylcarbonylaminoC₁-C₅alkyl, N—C₁-C₅alkylcarbonyl-N—C₁-C₅alkylaminoC₁-C₅alkyl, C₃-C₆-trialkylsilylC₁-C₅alkyl, phenylC₁-C₅alkyl (wherein the phenyl is optionally substituted by C₁-C₃alkyl, C₁-C₃haloalkyl, C₁-C₃alkoxy, C₁-C₃haloalkoxy, C₁-C₃alkylthio, C₁-C₃alkylsulfinyl, C₁-C₃alkylsulfonyl, halogen, cyano, or by nitro), heteroarylC₁-C₅alkyl (wherein the heteroaryl is optionally substituted by C₁-C₃alkyl, C₁-C₃haloalkyl, C₁-C₃alkoxy, C₁-C₃haloalkoxy, C₁-C₃alkylthio, C₁-C₃alkylsulfinyl, C₁-C₃alkylsulfonyl, halogen, cyano, or by nitro), C₃-C₈haloalkenyl, C₃-C₈cycloalkyl, phenyl or phenyl substituted by C₁-C₃alkyl, C₁-C₃haloalkyl, C₁-C₃alkoxy, C₁-C₃haloalkoxy, halogen, cyano or nitro, heteroaryl or heteroaryl substituted by C₁-C₃alkyl, C₁-C₃haloalkyl, C₁-C₃alkoxy, C₁-C₃haloalkoxy, halogen, cyano or nitro.

In the compound of formula (I), preferably R¹ is ethyl.

In the compound of formula (I), preferably R² is phenyl substituted by methyl, methoxy, or halogen, more preferably fluorine or chlorine, such as chlorine.

Still more preferably, R² is 4-chlorophenyl, 4-chloro-2-fluorophenyl, 2,4-dichlorophenyl, 4-chloro-2-methylphenyl, or 4-chloro-2-methoxyphenyl. Most preferably, R² is 4-chloro-2-fluorophenyl or 2,4-dichlorophenyl.

Preferred are those compounds of formula (I), wherein R⁴, R⁵, R⁶ and R⁷, independently of each other, are hydrogen or C₁-C₂ alkyl, more preferably methyl.

Preferably, R^{a} and R^{b} are C₁-C₆alkyl; more preferably methyl, ethyl, n-propyl, isopropyl or t-butyl; still more preferably methyl.

Preferably, G is hydrogen, C(O)—R^{a} or C(O)—O—R^{b}; wherein R^{a} and R^{b} are C₁-C₆alkyl, more preferably methyl, ethyl, n-propyl, isopropyl or t-butyl, still more preferably methyl.

In a particular embodiment, G is hydrogen.

When G is hydrogen, then the compound of formula (I) can be present as the first or the second of the two equilibrating tautomeric forms shown below or as a mixture of both tautomeric forms. The compound of formula (I), as used in the present invention, encompasses the first tautomeric form, the second tautomeric form, and a mixture of the first and second tautomeric forms:

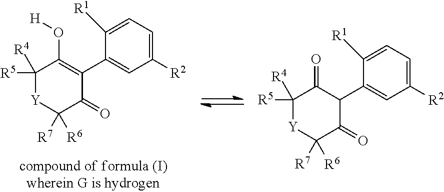

Preferably, the compound of formula (I) is:

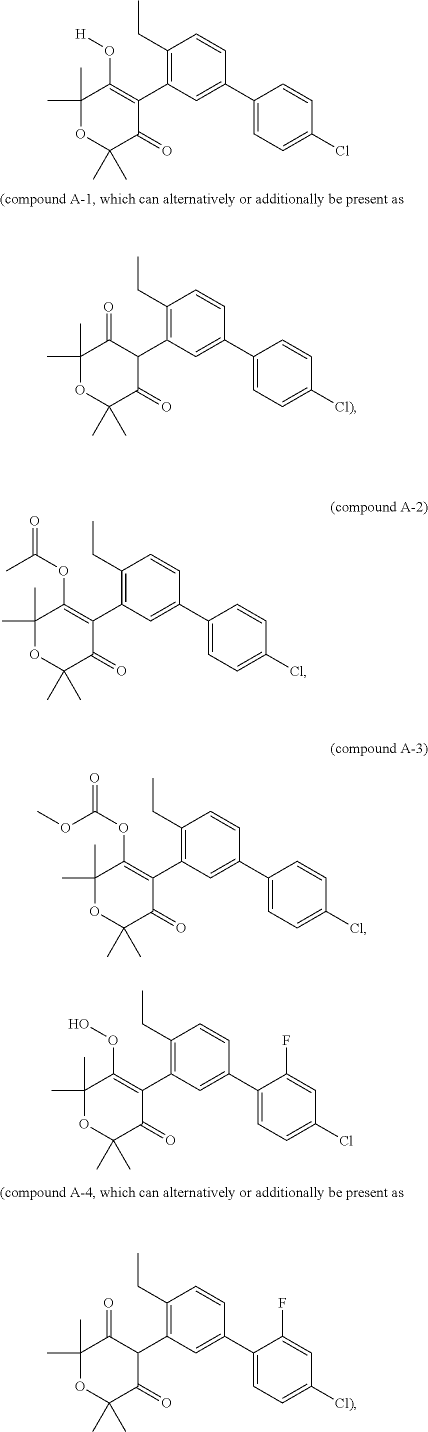

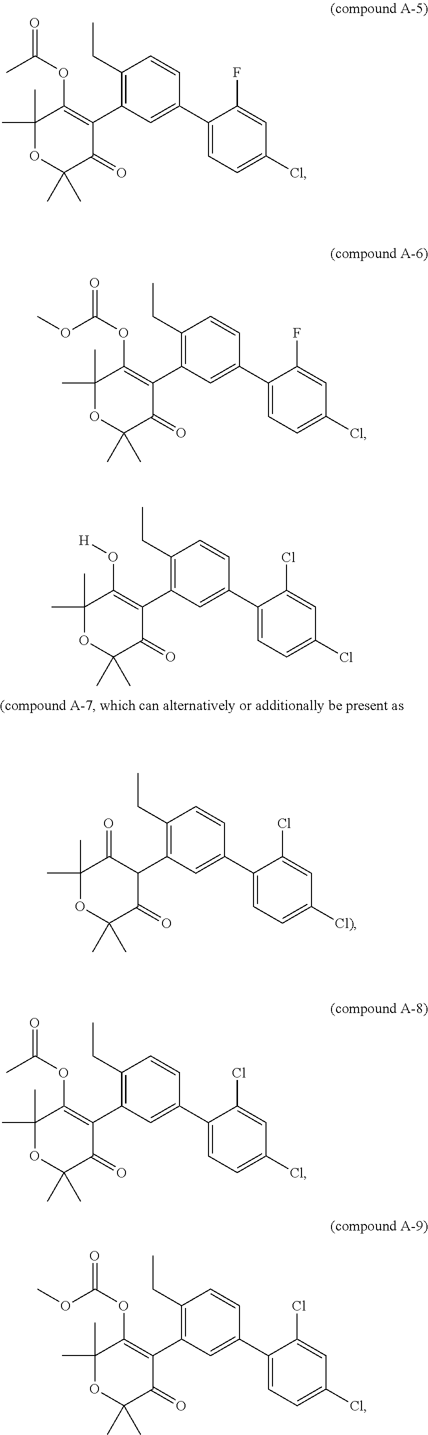

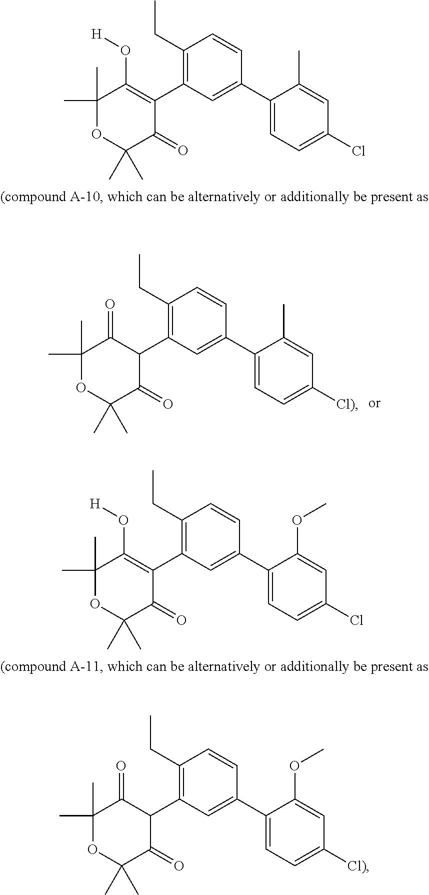

Preferably, the compound of formula (I) is one of compounds A-1 to A-9.

More preferably, the compound of formula (I) is compound A-4, A-5, A-6, A-7, A-8 or A-9.

Most preferably, the compound of formula (I) is compound A-4, A-7 or A-9.

Compounds A-4, A-7 and A-9, within the present invention, were previously disclosed as compounds A-66, A-45 and D-26 respectively in WO 2008/071405 A1, and can be synthesized using the methods of preparation disclosed therein.

In one preferred embodiment, the compound of formula (I) is compound A-4.

In another preferred embodiment, the compound of formula (I) is compound A-7.

In another preferred embodiment, the compound of formula (I) is compound A-9.

Preferred or particular or optional embodiments for the co-herbicide are as follows.

In one particular embodiment, the co-herbicide is ipfencarbazone. Ipfencarbazone, whose CAS (Chemical Abstracts Service) registry number is 212201-70-2, is a herbicide of the triazolone (or “tetrazolinone”) class, and is believed to be an inhibitor of very long chain fatty acids, which usually inhibit cell division, in plants. Ipfencarbazone has the following structure:

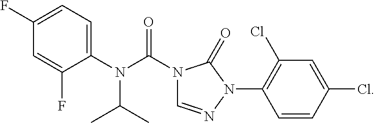 [SMILES: CC(C)N(C(=O)n1cnn(-c2ccc(Cl)cc2Cl)c1=O)c1ccc(F)cc1F]

Ipfencarbazone may also be in the form of a salt (e.g. agriculturally acceptable salt) thereof, and salts are encompassed within the meaning of ipfencarbazone. Ipfencarbazone is disclosed as Compound no. 231 in Table 1a on page 32 of EP 0 974 587 A1 (Hokko Chemical Industry Co. Ltd.) derived from WO 98/38176.

In the particular embodiment wherein the co-herbicide is ipfencarbazone, preferably, the compound of formula (I) is compound A-4, A-7 or A-9.

In one particular embodiment, the co-herbicide is propyrisulfuron. Propyrisulfuron (TH-547), whose CAS registry number is 570415-88-2. has the following structure:

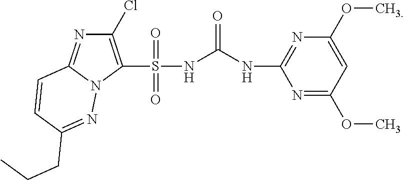 [SMILES: CCCc1ccc2nc(Cl)c(S(=O)(=O)NC(=O)Nc3nc(OC)cc(OC)n3)n2n1]

Propyrisulfuron may also be in the form of a salt (e.g. agriculturally acceptable salt) thereof, and salts are encompassed within the meaning of propyrisulfuron. Propyrisulfuron and its synthesis is disclosed as Compound no. 38 in Synthesis Example 4 on pages 44-45 of EP 1 466 527 A1 (Sumitomo Chemical Takeda Agro Company, Ltd) derived from WO 03/061388.

In the particular embodiment wherein the co-herbicide is propyrisulfuron, preferably, the compound of formula (I) is compound A-4, A-7 or A-9. This embodiment appears to have certain advantages, as disclosed in Biological Example 1 and the comments thereon hereinafter.

In one particular embodiment, the co-herbicide is N-[2-[(4,6-dimethoxy-1,3,5-triazin-2-yl)carbonyl]-6-fluorophenyl]-1,1-difluoro-N-methylmethanesulfonamide. This compound, whose CAS registry number is 874195-61-6, has the following structure:

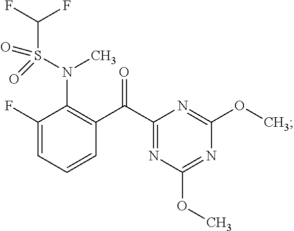 [SMILES: COc1nc(OC)nc(C(=O)c2cccc(F)c2N(C)S(=O)(=O)C(F)F)n1]

and is a herbicide of the keto sulfonanilide class, currently under development by Bayer CropScience AG. It is disclosed inter alia in WO 2006/008159 A1 (for fungicidal use; Bayer CropScience AG). Its proposed ISO common name is triafamone (SN 1211). It may also be in the form of a salt (e.g. agriculturally acceptable salt) thereof. Therefore, all references herein to N-[2-[(4,6-dimethoxy-1,3,5-triazin-2-yl)carbonyl]-6-fluorophenyl]-1,1-difluoro-N-methylmethanesulfonamide are intended to encompass the compound or a salt (e.g. an agriculturally acceptable salt) thereof.

However, preferably, the co-herbicide is fenoxasulfone. Fenoxasulfone, whose CAS registry number is 639826-16-7, is a herbicide of the 4,5-dihydro-1,2-oxazole (4,5-dihydro-isoxazole) class. Fenoxasulfone is likely to be an inhibitor of very long chain fatty acids, and/or is likely to inhibit cell division, in plants. Fenoxasulfone has the following structure:

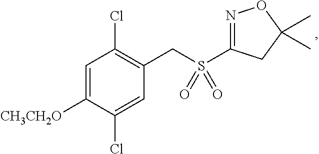

Fenoxasulfone may also be in the form of a salt (e.g. agriculturally acceptable salt) thereof, and salts are encompassed within the meaning of fenoxasulfone. Fenoxasulfone is covered by the scope of the isoxazoline herbicides disclosed in EP 1 203 768 A1 and CA 2 380 499 A1, both derived from WO 01/012613 A1 (Kumiai Chemical Industry Co., Ltd and Ihara Chemical Industry Co., Ltd). Mixtures of isoxazoline herbicides, e.g. specifically fenoxasulfone, with various other herbicides are disclosed in JP 2004/002324 A and JP 2005/145958 A (both publications Kumiai Chemical Industry Co., Ltd and Ihara Chemical Industry Co., Ltd). EP 2 135 508 A1 (Kumiai Chemical Industry Co., Ltd), derived from WO 2008/114493, discloses a herbicidal composition containing: (A) an isoxazoline derivative, for example fenoxasulfone (which is compound no. 54 on Table 2 on page 12), and (B) a cyclohexanedione type compound, a phenylpyrazoline type compound, a sulfonyl-aminocarbonyltriazolinone type compound, or a compound selected from a list of other co-herbicides such as pinoxaden.

More preferably, the compound of formula (I) is compound A-4, A-7 or A-9, and the co-herbicide is fenoxasulfone. This embodiment appears to have certain advantages, e.g. in flooded conditions, as disclosed in Biological Example 1 and the comments thereon hereinafter, such as generally good herbicidal activity in flooded conditions against both Echinichloa crus-galli (ECHCG) and Leptochloa chinensis (LEFCH) weeds, when compound A-4, A-7 or A-9 are added as an emulsifiable concentrate (EC) composition.

In one more preferred embodiment, the compound of formula (I) is compound A-4, and the co-herbicide is fenoxasulfone.

In another more preferred embodiment, the compound of formula (I) is compound A-7, and the co-herbicide is fenoxasulfone.

In another more preferred embodiment, the compound of formula (I) is compound A-9, and the co-herbicide is fenoxasulfone.

Preferably, the weight ratio of the compound of formula (I) (e.g. compound A-4, A-7 or A-9) to the fenoxasulfone is from 1:6 to 3:2 (e.g. 60:200, 90:200, 120:200, or 240:200); or more preferably is from 1:5 to 1:1 or is from 1:4 to 4:5 (e.g. 60:200, 90:200, or 120:200), or still more preferably is from 3:10 to 4:5 or is from 3:10 to 7:10 (e.g. 60:200, 90:200, or 120:200). Yet more preferably, the weight ratio of the compound of formula (I) (e.g. compound A-4, A-7 or A-9) to the fenoxasulfone is from 2:5 to 4:5 or is from 2:5 to 7:10 or is from 9:20 to 7:10 (e.g. 90:200 or 120:200). Most preferably, the weight ratio of the compound of formula (I) (e.g. compound A-4, A-7 or A-9) to the fenoxasulfone is from 1:2 to 7:10 (e.g. 120:200), preferably 3:5.

Preferably, the weight ratio of the compound of formula (I) (e.g. compound A-4, A-7 or A-9) to the ipfencarbazone is from 1:7 to 1:1 (e.g. 60:250, 90:250, 120:250, or 240:250), or more preferably is from 1:5 to 2:3 or is from 6:25 to 1:2 (e.g. 60:250, 90:250, or 120:250).

Preferably, the weight ratio of the compound of formula (I) (e.g. compound A-4, A-7 or A-9) to the propyrisulfuron is from 1:2 to 3:1 (e.g. 60:80, 90:80, 120:80, or 240:80), or more preferably is from 1:2 to 2:1 or is from 3:4 to 3:2 (e.g. 60:80, 90:80, or 120:80).

In one particular embodiment, the weight ratio of the compound of formula (I) (e.g. compound A-4, A-7 or A-9) to the N-[2-[(4,6-dimethoxy-1,3,5-triazin-2-yl)carbonyl]-6-fluorophenyl]-1,1-difluoro-N-methylmethanesulfonamide is from 1:20 to 20:1, e.g. from 1:10 to 10:1.

The herbicidal compositions of the present invention can be prepared in a variety of ways using formulation adjuvants, such as carriers (e.g. liquid or solid carrier), solvents and/or surface-active substances. Therefore, preferably, the herbicidal composition of the present invention is a formulation comprising a carrier (e.g. liquid or solid carrier), a solvent and/or a surface-active substance.

The formulation can be in various physical forms, for example in the form of a dustable powder (DP), a gel, a wettable powder (WP), a granule (GR) (such as an emulsifiable granule (EG) or more particularly a water-dispersible granule (WG)), a water-dispersible tablet (WT), an effervescent compressed tablet, an emulsifiable concentrate (EC), a micro-emulsifiable concentrate, an oil-in-water emulsion (EW), an oil flowable (e.g. a spreading oil (SO)), an aqueous dispersion (e.g. aqueous suspension concentrate (SC)), an oily dispersion (OD), a suspo-emulsion (SE), a capsule suspension (CS), a soluble liquid, a water-soluble concentrate (with water or a water-miscible organic solvent as carrier), an impregnated polymer film, or in another form such as a form known, for example, from the Manual on Development and Use of FAO Specifications for Plant Protection Products, 5th Edition, 1999.

Preferably, the formulation is in the form of a wettable powder (WP), a granule (GR) (such as an emulsifiable granule (EG) or more particularly a water-dispersible granule (VVG)), an emulsifiable concentrate (EC), a microemulsifiable concentrate, an oil-in-water emulsion (EW), an oil flowable (e.g. a spreading oil (SO)), an aqueous dispersion (e.g. aqueous suspension concentrate (SC)), an oily dispersion (OD), a soluble liquid, or a water-soluble concentrate (wherein the water-soluble concentrate is with water or a water-miscible organic solvent as carrier). More preferably, the formulation is in the form of an emulsifiable concentrate (EC).

Such formulations can either be used directly or can be diluted prior to use. Diluted formulations can be prepared, for example, by mixing with water, liquid fertiliser(s), micro-nutrient(s), biological organism(s), oil and/or solvent(s).

The formulations can be prepared, for example, by mixing the active ingredient with formulation adjuvants in order to obtain compositions in the form of finely divided solids, granules, solutions, dispersions or emulsions. The active ingredients can also be formulated with other adjuvants, for example finely divided solids, mineral oils, vegetable oils, modified vegetable oils, organic solvents, water, surface-active substances or combinations thereof. The active ingredients can also be contained in very fine microcapsules consisting of a polymer. Microcapsules contain the active ingredients in a porous carrier. This enables the active ingredients to be released into their surroundings in controlled amounts (e.g. slow release). Microcapsules usually have a diameter of from 0.1 to 500 microns. They contain active ingredients in an amount of about from 25 to 95% by weight of the capsule weight. The active ingredients can be present in the form of a monolithic solid, in the form of fine particles in solid or liquid dispersion or in the form of a suitable solution. The encapsulating membranes comprise, for example, natural and synthetic gums, cellulose, styrene-butadiene copolymers, polyacrylonitrile, polyacrylate, polyester, polyamides, polyureas, polyurethane or chemically modified polymers and starch xanthates or other polymers that are known to the person skilled in the art in this connection. Alternatively it is possible for very fine microcapsules to be formed wherein the active ingredient is present in the form of finely divided particles in a solid matrix of a base substance, but in that case the microcapsule is not encapsulated.

The formulation adjuvants suitable for the preparation of the compositions according to the invention can include those known per se.

As liquid carrier (and/or solvent) there may be used: water, toluene, xylene, petroleum ether, vegetable oils, acetone, methyl ethyl ketone, cyclohexanone, acid anhydrides, acetonitrile, acetophenone, amyl acetate, 2-butanone, butylenes carbonate, chlorobenzene, cyclo-hexane, cyclohexanol, alkyl esters of acetic acid (e.g. ethyl acetate, butyl acetate, amyl acetate, or isoamyl acetate, et al.), diacetone alcohol, 1,2-dichloropropane, diethanolamine, p-diethylbenzene, diethylene glycol, diethylene glycol abietate, diethylene glycol butyl ether, diethylene glycol ethyl ether, diethylene glycol methyl ether, N,N-dimethylformamide, dimethyl sulfoxide, 1,4-dioxane, dipropylene glycol, dipropylene glycol methyl ether, dipropylene glycol dibenzoate, diproxitol, alkylpyrrolidone, ethyl acetate, 2-ethyl-hexanol, ethylene carbonate, 1,1,1-trichloroethane, 2-heptanone, alpha-pinene, d-limonene, ethyl lactate, ethylene glycol, ethylene glycol butyl ether, ethylene glycol methyl ether, gamma-butyrolactone, glycerol, glycerol acetate, glycerol diacetate, glycerol triacetate, hexadecane, hexylene glycol, isoamyl acetate, isobornyl acetate, isooctane, isophorone, isopropylbenzene, isopropyl myristate, lactic acid, laurylamine, mesityl oxide, methoxypropanol, methyl isoamyl ketone, methyl isobutyl ketone, methyl laurate, methyl octanoate, methyl oleate, methylene chloride, m-xylene, n-hexane, n-octylamine, octadecanoic acid, octylamine acetate, oleic acid, oleylamine, o-xylene, phenol, polyethylene glycol (PEG 400), propionic acid, propyl lactate, propylene carbonate, propylene glycol, propylene glycol methyl ether, p-xylene, toluene, triethyl phosphate, triethylene glycol, xylenesulfonic acid, paraffin, mineral oil, trichloroethylene, perchloroethylene, ethyl acetate, amyl acetate, butyl acetate, propylene glycol methyl ether, diethylene glycol methyl ether, methanol, ethanol, isopropanol, or higher molecular weight alcohols (i.e. an alcohol having a higher molecular weight than isopropanol, such as amyl alcohol, tetrahydrofurfuryl alcohol, hexanol, octanol, 2-ethyl-hexanol, cydohexanol, ethylene glycol, propylene glycol, diethylene glycol, triethylene glycol, hexylene glycol, or glycerol), or N-methyl-2-pyrrolidone, N-octyl-2-pyrrolidone, a mixture of heavy aromatic hydrocarbons (e.g. containing a mixture of C₁-C₄alkylnaphthalenes and optionally also naphthalene, e.g. Solvesso 200™) or a similar liquid carrier (and/or a similar solvent).

Water is generally the carrier of choice for the dilution of a concentrate formulation.

Suitable solid carriers are, for example, talc, titanium dioxide, pyrophyllite clay, silica (silicon dioxide), attapulgite clay, kieselguhr, limestone, calcium carbonate, bentonite, calcium montomorillonite, cottonseed husks, wheatmeal, soybean flour, pumice, wood flour, ground walnut shells, lignin and/or similar materials, as described, for example, in CFR 180.1001. (c) & (d).

A large number of surface-active substances can advantageously be used both in solid and in liquid formulations, especially in those formulations which can be diluted with a carrier prior to use. Surface-active substances may be anionic, cationic, non-ionic or polymeric and they may be used as emulsifying, wetting or suspending agents or for other purposes. Typical surface-active substances include, for example, salts of alkyl sulfates, such as diethanolammonium lauryl sulfate; salts of alkylarylsulfonates, such as calcium dodecyl-benzenesulfonate; alkylphenol-alkylene oxide addition products, such as nonylphenol ethoxylate; alcohol-alkylene oxide addition products, such as tridecyl alcohol ethoxylate; soaps, such as sodium stearate; salts of alkylnaphthalenesulfonates, such as sodium dibutylnaphthalenesulfonate; dialkyl esters of sulfosuccinate salts, such as sodium di(2-ethylhexyl)sulfosuccinate; sorbitol esters, such as sorbitol oleate; quaternary amines, such as lauryl trimethylammonium chloride, polyethylene glycol esters of fatty acids, such as polyethylene glycol stearate; block copolymers of ethylene oxide and propylene oxide; and salts of mono- and di-alkyl phosphate esters; and also further substances described e.g. in “McCutcheon's Detergents and Emulsifiers Annual”, MC Publishing Corp., Ridgewood, N.J., 1981.

Further adjuvants which can usually be used in pesticidal formulations include crystallisation inhibitors, viscosity-modifying substances, suspending agents, dyes, anti-oxidants, foaming agents, light absorbers, mixing aids, anti-foams, complexing agents, neutralising or pH-modifying substances and buffers, corrosion-inhibitors, fragrances, wetting agents, absorption improvers, micronutrients, plasticisers, glidants, lubricants, dispersants, thickeners, anti-freezes, microbiocides, and also liquid and solid fertilisers.

The formulations may also comprise additional active substances, for example further herbicides, herbicide safeners, plant growth regulators, fungicides or insecticides.

The compositions according to the invention can additionally include an additive comprising an oil of vegetable or animal origin, a mineral oil, alkyl esters of such oils or mixtures of such oils and oil derivatives. The amount of oil additive used in the composition according to the invention is generally from 0.01 to 10%, based on the spray mixture. For example, the oil additive can be added to the spray tank in the desired concentration after the spray mixture has been prepared. Preferred oil additives comprise mineral oils or an oil of vegetable origin, for example rapeseed oil, olive oil or sunflower oil, emulsified vegetable oil, such as AMIGO® (Rhône-Poulenc Canada Inc.), alkyl esters of oils of vegetable origin, for example the methyl derivatives, or an oil of animal origin, such as fish oil or beef tallow. A preferred additive contains, for example, as active components essentially 80% by weight alkyl esters of fish oils and 15% by weight methylated rapeseed oil, and also 5% by weight of customary emulsifiers and pH modifiers. Especially preferred oil additives comprise alkyl esters of C₈-C₂₂ fatty acids, especially the methyl derivatives of C₁₂-C₁₈ fatty acids, for example the methyl esters of lauric acid, palmitic acid and oleic acid, being important. Those esters are known as methyl laurate (CAS-111-82-0), methyl palmitate (CAS-112-39-0) and methyl oleate (CAS-112-62-9). A preferred fatty acid methyl ester derivative is Emery® 2230 and 2231 (Cognis GmbH). Those and other oil derivatives are also known from the Compendium of Herbicide Adjuvants, 5th Edition, Southern Illinois University, 2000.

The application and action of the oil additives can be further improved by combining them with surface-active substances, such as non-ionic, anionic or cationic surfactants. Examples of suitable anionic, non-ionic and cationic surfactants are listed on pages 7 and 8 of WO 97/34485. Preferred surface-active substances are anionic surfactants of the dodecyl-benzylsulfonate type, especially the calcium salts thereof, and also non-ionic surfactants of the fatty alcohol ethoxylate type. Special preference is given to ethoxylated C₁₂-C₂₂ fatty alcohols having a degree of ethoxylation of from 5 to 40. Examples of commercially available surfactants are the Genapol types (Clariant AG). Also preferred are silicone surfactants, especially polyalkyl-oxide-modified heptamethyltrisiloxanes, which are commercially available e.g. as Silwet L-77®, and also perfluorinated surfactants. The concentration of surface-active substances in relation to the total additive is generally from 1 to 30% by weight. Examples of oil additives that comprise or consist of mixtures of oils or mineral oils or derivatives thereof with surfactants are Edenor ME SU®, Turbocharge® (a mixture of surfactant, 1-octanol and petroleum oil) (Syngenta AG, CH), and Actipron® (BP Oil UK Limited, GB).

The said surface-active substances may also be used in the formulations alone, that is to say without oil additives.

Furthermore, the addition of an organic solvent to the oil additive/surfactant mixture can contribute to a further enhancement of action. Suitable solvents are, for example, Solvesso® (ESSO) and Aromatic Solvent® (Exxon Corporation). The concentration of such solvents can be from 10 to 80% by weight of the total weight. Such oil additives, which may be in admixture with solvents, are described, for example, in U.S. Pat. No. 4,834,908. A commercially available oil additive disclosed therein is known by the name MERGE® (BASF Corporation). A further oil additive that is preferred according to the invention is SCORE® (Syngenta Crop Protection Canada.)

In addition to the oil additives listed above, in order to enhance the activity of the compositions according to the invention it is also possible for formulations of alkylpyrrolidones, (e.g. Agrimax®) to be added to the spray mixture. Formulations of synthetic latices, such as, for example, polyacrylamide, polyvinyl compounds or poly-1-p-menthene (e.g. Bond®, Courier® or Emerald®) can also be used. Solutions that contain propionic acid, for example Eurogkem Pen-e-trate®, can also be mixed into the spray mixture as activity-enhancing agents.

The herbicidal formulations generally contain:

(a) from 0.1 to 99% by weight, especially from 0.1 to 95% by weight, more especially from 0.5 to 60% by weight or from 1 to 40% by weight, of a compound of formula (I); and
(b) from 1 to 99.9% by weight (such as from 5 to 99.9%, or from 40 to 99.5%, or from 60% to 99%, by weight) of a formulation adjuvant(s) (e.g. a total of from 1 to 99.9%, e.g. from 5 to 99.9% or from 40 to 99.5%, by weight of the herbicidal composition/formulation, of: any carrier (e.g. liquid or solid carrier) (if present), any solvent (if present), any surface-active substance (if present), and any other formulation adjuvant(s) present).

The formulation adjuvant(s) preferably include(s) from 0 to 25% (e.g. from 1 to 25%) by weight of a surface-active substance.

Unless the context requires otherwise, in this specification, % by weight means % by weight of the herbicidal composition or formulation.

Whereas commercial products (e.g. liquid compositions/formulations) will preferably be formulated as concentrates, the end user will normally employ diluted formulations.

Preferred formulations have especially the following compositions:

(%=percent by weight of the herbicidal composition or formulation):

### Emulsifiable Concentrates:

active ingredient: 1 to 95%, in particular 1 to 60% (e.g. 1 to 40%) or 60 to 90%
surface-active agent: 1 to 30%, preferably 5 to 30% or 5 to 20%
liquid carrier (and/or solvent): 1 to 90% or 1 to 80%, in particular 1 to 35% or 35 to 90% (such as 35 to 80%)

### Dusts:

active ingredient: 0.1 to 10 preferably 0.1 to 5%
solid carrier: 99.9 to 90%, preferably 99.9 to 99%

### Suspension Concentrates:

active ingredient: 2 to 75% or 5 to 75%, preferably 10 to 50%
water: 94 to 24%, preferably 88 to 30%
surface-active agent: 1 to 40%, preferably 2 to 30%

### Wettable Powders:

active ingredient: 0.5 to 90%, preferably 1 to 80%
surface-active agent: 0.5 to 20%, preferably 1 to 15%
solid carrier: 5 to 95%, preferably 15 to 90%

### Granules:

active ingredient: 0.1 to 30%, preferably 0.1 to 15%
solid carrier: 99.5 to 70%, preferably 97 to 85%,
where the term “active ingredient” refers to the mixture of compound of formula (I) with a co-herbicide.

The following Examples further illustrate, but do not limit, the invention.

| F1. Emulsifiable concentrates | a) | b) | c) | d) |
|---|---|---|---|---|
| active ingredient | 5% | 10% | 25% | 50% |
| calcium dodecylbenzene- | 6% | 8% | 6% | 8% |
| sulfonate | | | | |
| castor oil polyglycol ether | 4% | — | 4% | 4% |
| (36 mol of ethylene oxide) | | | | |
| octylphenol polyglycol ether | — | 4% | — | 2% |
| (7-8 mol of ethylene oxide) | | | | |
| NMP (N-methylpyrrolidone) | — | — | 10% | 20% |
| aromatic hydrocarbon | 85% | 78% | 55% | 16% |
| mixture C₉-C₁₂ | | | | |

Emulsions of any desired concentration can be prepared from such concentrates by dilution with water.

| F2. Solutions | a) | b) | c) | d) |
|---|---|---|---|---|
| active ingredient | 5% | 10% | 50% | 90% |
| 1-methoxy-3-(3-methoxy- | — | 20% | 20% | — |
| propoxy)-propane | | | | |
| polyethylene glycol MW 400 | 20% | 10% | — | — |
| NMP (N-methylpyrrolidone) | — | — | 30% | 10% |
| aromatic hydrocarbon | 75% | 60% | — | — |
| mixture C₉-C₁₂ | | | | |

The solutions are suitable for application in the form of microdrops.

| F3. Wettable powders | a) | b) | c) | d) |
|---|---|---|---|---|
| active ingredient | 5% | 25% | 50% | 80% |
| sodium lignosulfonate | 4% | — | 3% | — |
| sodium lauryl sulfate | 2% | 3% | — | 4% |
| sodium diisobutylnaphthalene- | — | 6% | 5% | 6% |
| sulfonate | | | | |
| octylphenol polyglycol ether | — | 1% | 2% | — |
| (7-8 mol of ethylene oxide) | | | | |
| highly dispersed silicic acid | 1% | 3% | 5% | 10% |
| kaolin | 88% | 62% | 35% | — |

The active ingredient is thoroughly mixed with the adjuvants and the mixture is thoroughly ground in a suitable mill, yielding wettable powders which can be diluted with water to give suspensions of any desired concentration.

| F4. Coated granules | a) | b) | c) |
|---|---|---|---|
| active ingredient | 0.1% | 5% | 15% |
| highly dispersed silicic acid | 0.9% | 2% | 2% |
| inorganic carrier | 99.0% | 93% | 83% |
| (diameter 0.1-1 mm) | | | |
| e.g. CaCO₃ or SiO₂ | | | |

The active ingredient is dissolved in methylene chloride, the solution is sprayed onto the carrier and the solvent is subsequently evaporated off in vacuo.

| F5. Coated granules | a) | b) | c) |
|---|---|---|---|
| active ingredient | 0.1% | 5% | 15% |
| polyethylene glycol MW 200 | 1.0% | 2% | 3% |
| highly dispersed silicic acid | 0.9% | 1% | 2% |
| inorganic carrier | 98.0% | 92% | 80% |
| (diameter 0.1-1 mm) | | | |
| e.g. CaCO₃ or SiO₂ | | | |

The finely ground active ingredient is applied uniformly, in a mixer, to the carrier moistened with polyethylene glycol. Non-dusty coated granules are obtained in this manner.

| F6. Extruder granules | a) | b) | c) | d) |
|---|---|---|---|---|
| active ingredient | 0.1% | 3% | 5% | 15% |
| sodium lignosulfonate | 1.5% | 2% | 3% | 4% |
| carboxymethylcellulose | 1.4% | 2% | 2% | 2% |
| kaolin | 97.0% | 93% | 90% | 79% |

The active ingredient is mixed and ground with the adjuvants and the mixture is moistened with water. The resulting mixture is extruded and then dried in a stream of air.

| F7. Dusts | a) | b) | c) |
|---|---|---|---|
| active ingredient | 0.1% | 1% | 5% |
| talcum | 39.9% | 49% | 35% |
| kaolin | 60.0% | 50% | 60% |

Ready-to-use dusts are obtained by mixing the active ingredient with the carriers and grinding the mixture in a suitable mill.

| F8. Suspension concentrates | a) | b) | c) | d) |
|---|---|---|---|---|
| active ingredient | 3% | 10% | 25% | 50% |
| ethylene glycol | 5% | 5% | 5% | 5% |
| nonylphenol polyglycol ether | — | 1% | 2% | — |
| (15 mol of ethylene oxide) | | | | |
| sodium lignosulfonate | 3% | 3% | 4% | 5% |
| carboxymethylcellulose | 1% | 1% | 1% | 1% |
| 37% aqueous formaldehyde | 0.2% | 0.2% | 0.2% | 0.2% |
| solution | | | | |
| silicone oil emulsion | 0.8% | 0.8% | 0.8% | 0.8% |
| water | 87% | 79% | 62% | 38% |

The finely ground active ingredient is intimately mixed with the adjuvants, yielding a suspension concentrate from which suspensions of any desired concentration can be prepared by dilution with water.

The term “active ingredient” in the examples mentioned above refers to the mixture of compound of formula (I) with a co-herbicide.

The invention relates also to a method for the control (e.g. selective control) of grasses and weeds in crops of useful plants, which comprises treating the useful plants (e.g. flooded plants), or the area under cultivation (e.g. flooded area), or the locus (e.g. flooded locus) thereof, with a herbicidal composition according to this invention.

The invention also relates to a method of controlling grasses and weeds in crops of useful plants, which comprises applying a herbicidal composition of the invention as defined herein to the plants (e.g. flooded plants) or to the locus (e.g. flooded locus) thereof.

The invention also relates to a herbicidal composition as defined herein, for controlling grasses and weeds in crops of useful plants (e.g. flooded plants), especially in crops of rice (e.g. flooded rice). The grasses and weeds to be controlled can for example comprise Echinochloa and/or Leptochloa.

The crops of useful plants, e.g. in which the compositions according to the invention can be used, are in particular cereals, cotton, soybeans, sugar beet, sugar cane, plantation crops, rape (e.g. oilseed rape), maize or rice; or more particularly cotton, soybeans, sugar beet, rape (e.g. oilseed rape), or rice.

The crops of useful plants are preferably rice, in particular indica rice (such as IR-64, Ciherang, Pusa e.g. Pusa-1121, Jiayu 293, or NK-3325 hybrid); or japonica rice (such as Koshihikari, Arborio, or Liangyou peiju e.g. Liangyou peiju PS3100).

The rice is preferably flooded rice.

The rice can for example be direct-seeded (e.g. dry sown or wet-sown) rice; which can optionally be flooded (e.g. before the herbicidal composition is applied). However, preferably the rice is transplanted rice, which is typically flooded (e.g. before the herbicidal composition is applied).

Non-selective weed control might also be a possibility in some circumstances.

The grasses and/or weeds to be controlled may be monocotyledonous and/or dicotyledonous weeds, such as, for example, Setaria, Echinochloa (e.g. Echinochloa crus-galli), Leptochloa (e.g. Leptochloa chinensis), Scirpus, Monochoria, Brachiaria, Commelina, Cyperus, Sagittaria, Elatine, Lindernia, Ludwigia, Stellaria, Nasturtium, Agrostis, Digitaria, Avena, Sinapis, Lolium, Solanum, Bromus, Alopecurus, Sorghum, Rottboellia, Abutilon, Sida, Xanthium, Amaranthus, Chenopodium, Ipomoea, Chrysanthemum, Galium, Viola and/or Veronica; and/or Schoenoplectus.

Preferably, the grasses and/or weeds to be controlled comprise: monocotyledonous weeds, and/or grasses and/or weeds found in rice fields e.g. rice paddy fields; and/or preferably the grasses and/or weeds to be controlled comprise Echinochloa (e.g. Echinochloa crus-galli (ECHCG), Echinochloa oryzoides, Echinochloa colona or colonum (ECHCO), Echinochloa crus-pavonis, or Echinochloa oryzicola; or Echinochloa muricata or Echinochloa stagnina), Leptochloa (e.g. Leptochloa chinensis (LEFCH) or Leptochloa panicoides), Scirpus (e.g. Scirpus fluviatilis, Scirpus pendulus, Scirpus triangulatus, or one of many other Scirpus species), Monochoria (e.g. Monochoria vaginalis (MOOVA) or Monochoria korsakovii), Brachiaria, Commelina, Cyperus (e.g. Cyperus serotinus), Sagittaria, Elatine, Lindernia and/or Ludwigia; and/or Schoenoplectus (e.g. Schoenoplectus mucronatus or Schoenoplectus juncoides).

More preferably, the grasses and/or weeds to be controlled comprise Echinochloa (e.g. Echinochloa crus-galli (ECHCG), Echinochloa oryzoides, Echinochloa colona or colonum (ECHCO), Echinochloa crus-pavonis, or Echinochloa oryzicola; or Echinochloa muricata or Echinochloa stagnina) and/or Leptochloa (e.g. Leptochloa chinensis (LEFCH) or Leptochloa panicoides); most preferably Echinochloa crus-galli (ECHCG) and/or Leptochloa chinensis (LEFCH).

Still more preferably, the grasses and/or weeds to be controlled (e.g. comprising Echinochloa and/or Leptochloa) are in crops of flooded rice, especially crops of flooded transplanted rice.

The term “crops” is to be understood as also including crops that have been rendered tolerant to herbicides or classes of herbicides (for example ALS, GS, EPSPS, PPO, ACCase or HPPD inhibitors) as a result of conventional methods of breeding or genetic engineering. Examples of crop that have been rendered tolerant e.g. to imidazolinones, such as imazamox, by conventional methods of breeding are Clearfield® summer rape (Canola) or Clearfield® rice. Examples of crops that have been rendered tolerant to herbicides by genetic engineering methods include e.g. glyphosate-resistant or glufosinate-resistant maize or rice varieties, e.g. those commercially available under the trade names RoundupReady® (glyphosate-resistant maize or rice) or LibertyLink® (glufosinate-resistant maize or rice).

Crops are also to be understood as being those which have been rendered resistant to harmful insects by genetic engineering methods, for example Bt maize (resistant to European corn borer), Bt cotton (resistant to cotton boll weevil) and also Bt potatoes (resistant to Colorado beetle). Examples of Bt maize are the Bt-176 maize hybrids of NK® (Syngenta Seeds). The Bt toxin is a protein that is formed naturally by Bacillus thuringiensis soil bacteria. Examples of toxins and transgenic plants able to synthesise such toxins are described in EP-A-451 878, EP-A-374 753, WO 93/07278, WO 95/34656, WO 03/052073 and EP-A-427 529. Examples of transgenic plants that contain one or more genes which code for an insecticidal resistance and express one or more toxins are KnockOut® (maize), Yield Gard® (maize), NuCOTIN33B® (cotton), Bollgard® (cotton), NewLeaf® (potatoes), NatureGard® and Protexcta®. Plant crops and their seed material can be resistant to herbicides and at the same time also to insect feeding (“stacked” transgenic events). Seed can, for example, have the ability to express an insecticidally active Cry3 protein and at the same time be glyphosate-tolerant. The term “crops” is to be understood as also including crops obtained as a result of conventional methods of breeding or genetic engineering which contain so-called output traits (e.g. improved flavour, storage stability, nutritional content).

Areas under cultivation are to be understood as including land where the crop plants are already growing as well as land intended for the cultivation of those crop plants.

The rate of application of the herbicides (compound of formula (I) in admixture with the co-herbicide) may vary within wide limits, and for example can depend upon the nature of the soil, the method of application (pre- or post-emergence; seed dressing; application to the seed furrow; no tillage application etc.), the crop plant, the weed or grass to be controlled, the prevailing climatic conditions, and/or other factors governed by the method of application, the time of application and/or the target crop.

The mixture (herbicidal composition) according to the invention can for example be applied at an application rate of 1 to 4000 g of the mixture of herbicides (compound of formula (I) in admixture with the co-herbicide) per ha, especially from 5 to 1000 g/ha or 80 to 800 g/ha. “ha” means hectare.

In the method of controlling (or for the control of) grasses and weeds in crops of useful plants (e.g. rice) and/or in the herbicidal composition for controlling grasses and weeds in crops of useful plants (e.g. rice), according to the invention, preferably, the herbicidal composition is applied to the plants or to the locus thereof at an application rate of 30 to 240 g of the compound of formula (I) (e.g. compound A-4, A-7 or A-9) per hectare, calculated as the weight of the compound of formula (I) excluding the weight of any optional counterions thereof. More preferably, the herbicidal composition is applied to the plants or to the locus thereof at an application rate of 50 to 150 g of the compound of formula (I) (e.g. compound A-4, A-7 or A-9) per hectare, calculated as the weight of the compound of formula (I) excluding the weight of any optional counterions thereof. Still more preferably, the herbicidal composition is applied to the plants or to the locus thereof at an application rate of 60 to 125 g (e.g. 60 g, 90 g, or 120 g) of the compound of formula (I) (e.g. compound A-4, A-7 or A-9) per hectare, calculated as the weight of the compound of formula (I) excluding the weight of any optional counterions thereof. Most preferably, the herbicidal composition is applied to the plants or to the locus thereof at an application rate of 90 to 125 g, or 90 to 120 g (e.g. 90 g or 120 g), of the compound of formula (I) (e.g. compound A-4, A-7 or A-9) per hectare, calculated as the weight of the compound of formula (I) excluding the weight of any optional counterions thereof.

In the method of controlling (or for the control of) grasses and weeds in crops of useful plants (e.g. rice) and/or in the herbicidal composition for controlling grasses and weeds in crops of useful plants (e.g. rice), according to the invention, preferably, the co-herbicide is fenoxasulfone, ipfencarbazone, or propyrisulfuron; and
when the co-herbicide is fenoxasulfone, then the herbicidal composition is applied to the plants or to the locus thereof at an application rate of 100 to 400 g (more preferably 120 to 400 g, or 150 g to 300 g, still more preferably 175 g to 250 g, most preferably 200 g) of fenoxasulfone per hectare, calculated as the weight of fenoxasulfone excluding the weight of any optional counterions thereof; and
when the co-herbicide is ipfencarbazone, then the herbicidal composition is applied to the plants or to the locus thereof at an application rate of 120 to 500 g (more preferably 180 g to 320 g, most preferably 250 g) of ipfencarbazone per hectare, calculated as the weight of ipfencarbazone excluding the weight of any optional counterions thereof; and
when the co-herbicide is propyrisulfuron, then the herbicidal composition is applied to the plants or to the locus thereof at an application rate of 40 to 160 g (more preferably 60 g to 120 g, still more preferably 60 g to 100 g, most preferably 80 g) of propyrisulfuron per hectare, calculated as the weight of propyrisulfuron excluding the weight of any optional counterions thereof.

A further aspect of the invention provides a method of controlling Leptochloa weeds (e.g. Leptochloa chinensis (LEFCH), and/or e.g. flooded Leptochloa weeds) in crops of useful plants (e.g. rice, e.g. flooded and/or transplanted rice), which comprises applying compound A-9, whose structure is

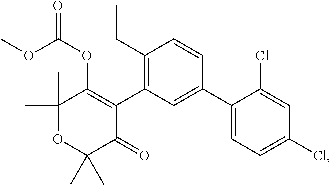 [SMILES: CCc1ccc(-c2ccc(Cl)cc2Cl)cc1C1=C(OC(=O)OC)C(C)(C)OC(C)(C)C1=O]

to the plants or to the locus (e.g. flooded locus) thereof. This method can use an application rate of 50-150 g/ha, e.g. 60-120 g/ha, of the compound A-9. This compound A-9 alone appears to be highly herbicidally active (80-90%) against flooded LEFCH, with little (5%) phytotoxicity on flooded transplanted IR-64 indica rice, when applied as an EC formulation at an application rate of 60, 90 or 120 g/ha (see Biological Example 1).

The compounds of formula (I) are generically disclosed in WO 2008/071405 A1 (Syngenta Participations AG and Syngenta Limited), and certain specific compounds of formula (I) (e.g. compounds A-4, A-7 and A-9, et al.) are specifically disclosed in WO 2008/071405 A1.

The mixing partners (co-herbicides, e.g. fenoxasulfone, ipfencarbazone, or propyrisulfuron) for the compound of formula (I), may also be in the form of salts (e.g. agriculturally acceptable salts).

Preferred herbicidal compositions comprise fenoxasulfone as co-herbicide. Another group of preferred compositions comprise ipfencarbazone as co-herbicide. A further group of preferred compositions comprise propyrisulfuron as co-herbicide.

The herbicidal compositions according to the invention can also be used in combination with safeners. The following mixtures with safeners, especially, come into consideration:

compound of formula (I)+cloquintocet-mexyl, compound of formula (I)+cloquintocet acid and salts thereof, compound of formula (I)+fenchlorazole-ethyl, compound of formula (I)+fenchlorazole acid and salts thereof, compound of formula (I)+mefenpyr-diethyl, compound of formula (I)+mefenpyr diacid, compound of formula (I)+isoxadifen-ethyl, compound of formula (I)+isoxadifen acid, compound of formula (I)+furilazole, compound of formula (I)+furilazole R isomer, compound of formula (I)+benoxacor, compound of formula (I)+dichlormid, compound of formula (I)+AD-67, compound of formula (I)+oxabetrinil, compound of formula (I)+cyometrinil, compound of formula (I)+cyometrinil Z-isomer, compound of formula (I)+fenclorim, compound of formula (I)+cyprosulfamide, compound of formula (I)+naphthalic anhydride, compound of formula (I)+flurazole, compound of formula (I)+CL 304,415, compound of formula (I)+dicyclonon, compound of formula (I)+fluxofenim, compound of formula (I)+DKA-24, compound of formula (I)+R-29148 and compound of formula (I)+PPG-1292. A safening effect can also be observed for the mixtures compound of the formula (I)+dymron, compound of the formula (I)+MCPA, compound of the formula (I)+mecoprop and compound of the formula (I)+mecoprop-P.

The above-mentioned safeners and herbicides are described, for example, in the Pesticide Manual, Twelfth Edition, British Crop Protection Council, 2000. R-29148 is described, for example by P. B. Goldsbrough et al., Plant Physiology, (2002), Vol. 130 pp. 1497-1505 and references therein and PPG-1292 is known from WO09211761.

The rates of application of the herbicide mixture are generally from 0.001 to 2 kg/ha, but preferably from 0.005 to 1 kg/ha.

The ratio by weight of the compound of formula (I) to the co-herbicide in the composition according to the invention is in particular from 1:20 to 20:1, more particularly from 1:10 to 10:1. See elsewhere herein for more specific weight ratios for specific co-herbicides (e.g. fenoxasulfone, ipfencarbazone or propyrisulfuron).

The rate of application of safener in relation to herbicide depends largely on the method of application. In the case of field treatment, which is effected either using a tank mixture comprising a combination of safener and herbicide mixture or by separate application of safener and herbicide mixture, the ratio of herbicides to safener is generally from 100:1 to 1:10, preferably from 20:1 to 1:1. In the case of field treatment, from 0.001 to 1.0 kg of safener/ha, preferably from 0.001 to 0.25 kg of safener/ha, is generally applied.

In the composition according to the invention, the amounts of oil additive employed are generally from 0.01 to 2%, based on the spray mixture. The oil additive can, for example, be added to the spray tank in the desired concentration after the spray mixture has been prepared.

Some non-limiting examples of the present invention are disclosed in the following Formulation Examples and/or Biological Examples.

### FORMULATION EXAMPLES

### Formulation Example 1

### Emulsifiable Concentrate (EC) Formulation of Compound A-4 (EC050 Formulation)

| Ingredient (chemical | | | | |
|---|---|---|---|---|
| structure/name or trade | | | | Concentration |
| name) | Role | Chemical name | Grade | (g · l⁻¹) |
| 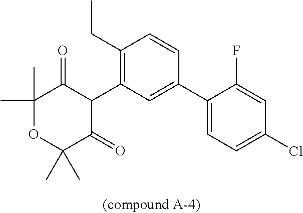 [SMILES: CCc1ccc(-c2ccc(Cl)cc2F)cc1C1C(=O)C(C)(C)OC(C)(C)C1=O] | Active ingredient (Al) | | | 50 |
| Emulsogen EL 360 ™ | Emulsifier | cator oil | 36 moles of | 95 |
| | | ethoxylate | ethylene oxide | |
| Nansa EVM 63/B ™ | Emulsifier | calcium salt of | 63% in | 63.3 |
| | | dodecyl-benzene | isobutanol | |
| | | sulfonic acid | | |
| Soprophor BSU ™ | Emulsifier | tristyrylphenol | 16 moles of | 31.7 |
| | | ethoxylate | ethylene oxide | |
| N-octyl-2-pyrrolidone | Solvent | N-octyl-2- | | 211.1 |
| | | pyrrolidone | | |
| 2-ethyl-hexanol | Solvent | 2-ethyl hexanol | | to 1 liter |

### Formulation Example 2

### Emulsifiable Concentrate (EC) Formulation of Compound A-7 (EC050 Formulation)

| Ingredient (chemical | | | | |
|---|---|---|---|---|
| structure/name or trade | | | | Concentration |
| name) | Role | Chemical name | Grade | (g · l⁻¹) |
| 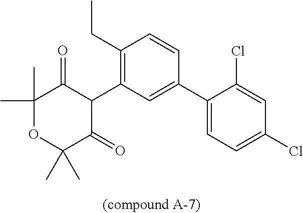 [SMILES: CCc1ccc(-c2ccc(Cl)cc2Cl)cc1C1C(=O)C(C)(C)OC(C)(C)C1=O] | Active ingredient (Al) | | | 50 |
| Emulsogen EL 360 ™ | Emulsifier | castor oil | 36 moles of | 94.7 |
| | | ethoxylate | ethylene oxide | |
| Nansa EVM 63/B ™ | Emulsifier | calcium salt of | 63% in | 63.1 |
| | | dodecyl-benzene | isobutanol | |
| | | sulfonic acid | | |
| Soprophor BSU ™ | Emulsifier | tristyrylphenol | 16 moles of | 31.6 |
| | | ethoxylate | ethylene oxide | |
| N-octyl-2-pyrrolidone | Solvent | N-octyl-2- | | 210.5 |
| | | pyrrolidone | | |
| 2-ethyl hexanol | Solvent | 2-ethyl hexanol | | to 1 litre |

### Formulation Example 3

### Emulsifiable Concentrate (EC) Formulation of Compound A-9 (EC050 Formulation)

| Ingredient (chemical | | | | |
|---|---|---|---|---|
| structure/name or | | | | Concentration |
| trade name) | Role | Chemical name | Grade | (g · l⁻¹) |
| 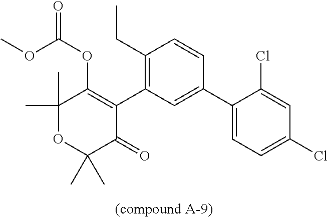 [SMILES: CCc1ccc(-c2ccc(Cl)cc2Cl)cc1C1=C(OC(=O)OC)C(C)(C)OC(C)(C)C1=O] | Active ingredient (Al) | | | 50 |
| Emulsogen EL 360 ™ | Emulsifier | castor oil | 36 moles of | 84.4 |
| | | ethoxylate | ethylene oxide | |
| Nansa EVM63/B ™ | Emulsifier | calcium salt of | 63% in | 42.2 |
| | | dodecyl- | isobutanol | |
| | | benzene | | |
| | | sulfonic acid | | |
| acetophenone | Solvent | methyl- | | 102.9 |
| | | phenylketone | | |
| Solvesso 200 ™ | Solvent | mixture of | ultra low | to 1 litre |
| | | heavy aromatic | naphthalene | |
| | | hydrocarbons | content | |

### Emulsifiable Concentrate (EC) Procedure (Used for Formulation Examples 1, 2 and 3)

The solvent(s) are added to the vessel, followed by the emulsifiers. The mixture is rolled until a clear solution is obtained. The active ingredient is then added, and rolled till a clear solution is obtained.

### Formulation Example 4

### Emulsifiable Concentrate (EC) Formulation Containing a Compound A-4 and a Co-Herbicide

Formulation Example 4 is a variation of Formulation Example 1 in which, in the emulsifiable concentrate (EC), in addition to compound A-4, there is present one co-herbicide selected from the group consisting of fenoxasulfone, ipfencarbazone, and propyrisulfuron, and wherein compound A-4 and the one co-herbicide are present in the following weight ratios:

Formulation Example 4A: weight ratio of compound A-4 to fenoxasulfone is: 60:200, or 90:200, or 120:200, or 240:200.
Formulation Example 4B: weight ratio of compound A-4 to ipfencarbazone is: 60:250, or 90:250, or 120:250; or 240:250.
Formulation Example 4C: weight ratio of compound A-4 to propyrisulfuron is: 60:80, or 90:80, or 120:80, or 240:80.

### Formulation Example 5

### Emulsifiable Concentrate (EC) Formulation Containing a Compound A-7 and a Co-Herbicide

Formulation Example 5 is a variation of Formulation Example 2 in which, in the emulsifiable concentrate (EC), in addition to compound A-7, there is present one co-herbicide selected from the group consisting of fenoxasulfone, ipfencarbazone, and propyrisulfuron, and wherein compound A-7 and the one co-herbicide are present in the following weight ratios:

Formulation Example 5A: weight ratio of compound A-7 to fenoxasulfone is: 60:200, or 90:200, or 120:200, or 240:200.
Formulation Example 5B: weight ratio of compound A-7 to ipfencarbazone is: 60:250, or 90:250, or 120:250, or 240:250.
Formulation Example 5C: weight ratio of compound A-7 to propyrisulfuron is: 60:80, or 90:80, or 120:80, or 240:80.

### Formulation Example 6

### Emulsifiable Concentrate (EC) Formulation Containing a Compound A-9 and a Co-Herbicide

Formulation Example 6 is a variation of Formulation Example 3 in which, in the emulsifiable concentrate (EC), in addition to compound A-9, there is present one co-herbicide selected from the group consisting of fenoxasulfone, ipfencarbazone, and propyrisulfuron, and wherein compound A-9 and the one co-herbicide are present in the following weight ratios:

Formulation Example 6A: weight ratio of compound A-9 to fenoxasulfone is: 60:200, or 90:200, or 120:200, or 240:200.
Formulation Example 6B: weight ratio of compound A-9 to ipfencarbazone is: 60:250, or 90:250, or 120:250, or 240:250.
Formulation Example 6C: weight ratio of compound A-9 to propyrisulfuron is: 60:80, or 90:80, or 120:80, or 240:80.

### Reference Formulation Example 7

### Suspension Concentrate (SC) Formulation of Pyroxasulfone—SC050 Formulation

| Ingredient | | | | |
|---|---|---|---|---|
| (chemical | | | | Concen- |
| structure/name | | | | tration |
| or trade name) | Role | Chemical name | Grade | (g · l⁻¹) |
| pyroxasulfone | Active | pyroxasulfone | | 50 |
| | ingredient | | | |
| | (Al) | | | |
| BORRESPERSE | Dispersant | sodium | | 35 |
| NA ™ | | ligninsulfonate | | |
| RHODOPOL | Thickener | xanthan gum | | 3.9 |
| 23 ™ | | | | |
| 1,2-propylene | Antifreeze | 1,2-propylene glycol | | 21 |
| glycol | | | | |
| PROXEL | Preservative | 1,2-benzisothiazol-3- | | 2.3 |
| GXL ™ | | one solution | | |
| SOPROPHOR | Dispersant | tristyrylphenol | | 3.5 |
| BSU ™ | | ethoxylate, with 16 | | |
| | | moles of ethylene | | |
| | | oxide (EO) | | |
| RHODORSIL | Antifoam | polydimethylsiloxane | | 2 |
| AF-454 ™ | | | | |
| Water | carrier | | | Remainder |

### Suspension Concentrate (SC) Formulation Procedure

All the inert (i.e. non-herbicidal) substances are added to water, and are mixed until homogeneous. Then the active ingredient (here, pyroxasulfone) is added. The mixture is then subjected to high shear mixing to break up very large particles to a size that is suitable for milling. This pre-mix is milled in a bead mill (shaker mill), until the median particle size (D50) reaches less than 5 microns.

### BIOLOGICAL EXAMPLES

The test plants are grown in a greenhouse, simulating two main groups of rice cropping systems: direct seeded rice and transplanted rice (refer to S. K. De Datta (1981), Principle and Practices of Rice Production, John Wiley, New York for definitions of rice cropping systems). Plant preparation and chemical application are different in the two systems and examples of these are described in more detail in Biological Examples 1 and 2 below.

### Biological Example 1

### Method for Testing, and Results for, Mixtures of Compounds of Formula (I) and Co-Herbicides in Flooded Transplanted Rice and in Flooded Leptochloa Chinensis and Echinochloa crus-Galli (Test 125)

Rice seeds, variety IR-64 (an indica type of rice), were sown in seed trays. After 7 days the resulting plants were transplanted as 3 groups of 2 plants, into pots containing a standard sandy loam soil saturated with water replicating swampy conditions. These were grown on for a further 9 days in a glasshouse bay (30/20° C. day/night; 18/6 hours light/dark; 75% humidity). Leptochloa chinensis (LEFCH) and Echinochloa crus-galli (ECHCG) were sown as 2 separate groups of approx 10-20 seeds into pots 13 days prior to application of the test substances, in the same glasshouse conditions as the rice. Therefore, each pot of weeds contained a group of LEFCH and a separate group of ECHCG. All pots of rice and all pots of weeds were flooded to 2-3 cm water depth the day prior to application of the test substances. Growth stages at time of application were as follows: Rice: from 2 leaves on the main stem up to tillering; ECHCG: from 2 leaves on the main stem up to tillering; LEFCH: 2-3 leaves.

Test solutions were prepared by mixing the appropriate aliquots of the test substances in deionised water to give the desired treatment concentration. Most of the test substances were used as formulated products. Specifically, compound A-4 was applied as the EC050 formulation disclosed in Formulation Example 1 herein (EC=emulsifiable concentrate). Compound A-7 was applied as the EC050 Formulation disclosed in Formulation Example 2 herein. Compound A-9 was applied as the EC050 Formulation disclosed in Formulation Example 3 herein. Pyroxasulfone (a comparator co-herbicide) was applied as the SC050 formulation disclosed in Reference Formulation Example 7 herein (SC=Suspension concentrate). Imazosulfuron (a comparator co-herbicide) was applied as a GR0.25 formulation (GR=granule).

The test substances which were pure active ingredient (e.g. propyrisulfuron & fenoxasulfone) (labelled as ‘Technical’ in the following results table), were prepared by dissolving in 10.56% Emulsogen EL™ (castor oil ethoxylate, CAS Registry number 61791-12-6), 42.22% N-methylpyrrolidone and 42.22% dipropylene glycol mono-ethyl ether to give a stock solution containing 5% of the test substance and 95% of (Emulsogen EL™, N-methylpyrrolidone and dipropylene glycol mono-ethyl ether).

Application of the test substances was made by pipetting the required amount of the test solution or the test formulation gently into the flood water of the appropriate pot.

The test plants were then grown on in the same glasshouse conditions, and watered twice daily keeping the flood water at a depth of 2-3 cm.

A visual assessment of the % herbicidal damage was made 7 & 14 days after application (DAA), and the results are presented herein as % visual herbicidal damage where 0=no damage to plants and 100=total kill.

### Biological Example 1

### Results Summary Table for Flooded Transplanted Rice and Flooded ECHCG and LEFCH at 14 Days after Application (14 DAA) of Mixtures of Compounds of Formula (I) with Co-Herbicides

| | | compound | | | | |
|---|---|---|---|---|---|---|
| Compound of | | of formula | Co- | % | % | |
| formula (I) | Co-herbicide/ | (I) | herbicide | damage | damage | % damage |
| (and | mixture partner | application | application | to Rice | to | to |
| formulation) | (and formulation) | rate (g/ha) | rate (g/ha) | IR-64 | ECHCG | LEFCH |
| COMPOUND | | | | | | |
| A-4 | | | | | | |
| compound A-4 | none | 60 | — | | 55 | 0 |
| (EC050 | | | | | | |
| Formulation | | | | | | |
| Example 1) | | | | | | |
| compound A-4 | none | 90 | — | 0 | 85 | 0 |
| (EC050) | | | | | | |
| compound A-4 | none | 120 | — | 0 | 90 | 20 |
| (EC050) | | | | | | |
| compound A-4 | none | 240 | — | 10 | | |
| (EC050) | | | | | | |
| compound A-4 | imazosulfuron | 60 | 80 | | 65 | 0 |
| (EC050 | (GR0.25 | | | | | |
| Formulation | formulation) | | | | | |
| Example 1) | | | | | | |
| compound A-4 | imazosulfuron | 90 | 80 | 10 | 70 | 0 |
| (EC050) | (GR0.25) | | | | | |
| compound A-4 | imazosulfuron | 120 | 80 | 10 | 75 | 0 |
| (EC050) | (GR0.25) | | | | | |
| compound A-4 | imazosulfuron | 240 | 80 | 15 | | |
| (EC050) | (GR0.25) | | | | | |
| compound A-4 | propyrisulfuron | 60 | 80 | | 85 | 0 |
| (EC050 | (TH-547) | | | | | |
| Formulation | (Technical) | | | | | |
| Example 1) | | | | | | |
| compound A-4 | propyrisulfuron | 90 | 80 | 5 | 85 | 0 |
| (EC050) | (Technical) | | | | | |
| compound A-4 | propyrisulfuron | 120 | 80 | M | 85 | 0 |
| (EC050) | (Technical) | | | | | |
| compound A-4 | propyrisulfuron | 240 | 80 | 10 | | |
| (EC050) | (Technical) | | | | | |
| compound A-4 | fenoxasulfone | 60 | 200 | | 95 | 95 |
| (EC050 | (Technical) | | | | | |
| Formulation | | | | | | |
| Example 1) | | | | | | |
| compound A-4 | fenoxasulfone | 90 | 200 | 10 | 90 | 95 |
| (EC050) | (Technical) | | | | | |
| compound A-4 | fenoxasulfone | 120 | 200 | 25 | 99 | 95 |
| (EC050) | (Technical) | | | | | |
| compound A-4 | fenoxasulfone | 240 | 200 | 35 | | |
| (EC050) | (Technical) | | | | | |
| COMPOUND | | | | | | |
| A-7 | | | | | | |
| compound A-7 | none | 60 | — | | 35 | 0 |
| (EC050 | | | | | | |
| Formulation | | | | | | |
| Example 2) | | | | | | |
| compound A-7 | none | 90 | — | 5 | 55 | 5 |
| (EC050) | | | | | | |
| compound A-7 | None | 120 | — | 10 | 65 | 5 |
| (EC050) | | | | | | |
| compound A-7 | None | 240 | — | 10 | | |
| (EC050) | | | | | | |
| compound A-7 | imazosulfuron | 60 | 80 | | 35 | 0 |
| (EC050 | (GR0.25 | | | | | |
| Formulation | formulation) | | | | | |
| Example 2) | | | | | | |
| compound A-7 | imazosulfuron | 90 | 80 | 15 | 45 | 0 |
| (EC050) | (GR0.25) | | | | | |
| compound A-7 | imazosulfuron | 120 | 80 | 15 | 60 | 0 |
| (EC050) | (GR0.25) | | | | | |
| compound A-7 | imazosulfuron | 240 | 80 | 25 | | |
| (EC050) | (GR0.25) | | | | | |
| compound A-7 | propyrisulfuron | 60 | 80 | | 65 | 10 |
| (EC050 | (TH-547) | | | | | |
| Formulation | (Technical) | | | | | |
| Example 2) | | | | | | |
| compound A-7 | propyrisulfuron | 90 | 80 | 5 | 70 | 10 |
| (EC050) | (Technical) | | | | | |
| compound A-7 | propyrisulfuron | 120 | 80 | 5 | 65 | 10 |
| (EC050) | (Technical) | | | | | |
| compound A-7 | propyrisulfuron | 240 | 80 | 5 | | |
| (EC050) | (Technical) | | | | | |
| compound A-7 | pyroxasulfone | 60 | 10 | | 60 | M |
| (EC050 | (SC050, Reference | | | | | |
| Formulation | Formulation | | | | | |
| Example 2) | Example 7) | | | | | |
| compound A-7 | pyroxasulfone | 90 | 10 | 45 | 70 | M |
| (EC050) | (SC050) | | | | | |
| compound A-7 | pyroxasulfone | 120 | 10 | 45 | 80 | 10 |
| (EC050) | (SC050) | | | | | |
| compound A-7 | pyroxasulfone | 240 | 10 | 45 | | |
| (EC050) | (SC050) | | | | | |
| compound A-7 | fenoxasulfone | 60 | 200 | | 65 | 70 |
| (EC050 | (Technical) | | | | | |
| Formulation | | | | | | |
| Example 2) | | | | | | |
| compound A-7 | fenoxasulfone | 90 | 200 | 20 | 70 | 70 |
| (EC050) | (Technical) | | | | | |
| compound A-7 | fenoxasulfone | 120 | 200 | 25 | 70 | 90 |
| (EC050) | (Technical) | | | | | |
| compound A-7 | fenoxasulfone | 240 | 200 | 20 | | |
| (EC050) | (Technical) | | | | | |
| COMPOUND | | | | | | |
| A-9 | | | | | | |
| compound A-9 | none | 60 | — | | 15 | 80 |
| (EC050 | | | | | | |
| Formulation | | | | | | |
| Example 3) | | | | | | |
| compound A-9 | none | 90 | — | 5 | 40 | 80 |
| (EC050) | | | | | | |
| compound A-9 | none | 120 | — | 5 | 70 | 90 |
| (EC050) | | | | | | |
| compound A-9 | none | 240 | — | 15 | | |
| (EC050) | | | | | | |
| compound A-9 | imazosulfuron | 60 | 80 | | 45 | 90 |
| (EC050 | (GR0.25 | | | | | |
| Formulation | formulation) | | | | | |
| Example 3) | | | | | | |
| compound A-9 | imazosulfuron | 90 | 80 | 0 | 50 | M |
| (EC050) | (GR0.25) | | | | | |
| compound A-9 | imazosulfuron | 120 | 80 | M | 50 | 90 |
| (EC050) | (GR0.25) | | | | | |
| compound A-9 | imazosulfuron | 240 | 80 | 5 | | |
| EC050 | (GR0.25) | | | | | |
| compound A-9 | propyrisulfuron | 60 | 80 | | 75 | 70 |
| (EC050 | (TH-547) | | | | | |
| Formulation | (Technical) | | | | | |
| Example 3) | | | | | | |
| compound A-9 | propyrisulfuron | 90 | 80 | 10 | 75 | M |
| (EC050) | (Technical) | | | | | |
| compound A-9 | propyrisulfuron | 120 | 80 | 10 | 80 | 70 |
| (EC050) | (Technical) | | | | | |
| compound A-9 | propyrisulfuron | 240 | 80 | 15 | | |
| (EC050) | (Technical) | | | | | |
| compound A-9 | fenoxasulfone | 60 | 200 | | 60 | 90 |
| (EC050 | (Technical) | | | | | |
| Formulation | | | | | | |
| Example 3) | | | | | | |
| compound A-9 | fenoxasulfone | 90 | 200 | 5 | 70 | 70 |
| (EC050) | (Technical) | | | | | |
| compound A-9 | fenoxasulfone | 120 | 200 | 5 | 90 | 85 |
| (EC050) | (Technical) | | | | | |
| compound A-9 | fenoxasulfone | 240 | 200 | 5 | | |
| (EC050) | (Technical) | | | | | |
| CO- | | | | | | |
| HERBICIDES | | | | | | |
| ALONE | | | | | | |
| None | imazosulfuron | — | 80 | 0 | 50 | 0 |
| | (GR0.25 | | | | | |
| | formulation) | | | | | |
| none | propyrisulfuron | — | 80 | 0 | 80 | 0 |
| | (TH-547) | | | | | |
| | (Technical) | | | | | |
| none | pyroxasulfone | — | 10 | 45 | 60 | 70 |
| | (SC050, Reference | | | | | |
| | Formulation | | | | | |
| | Example 7) | | | | | |
| none | fenoxasulfone | — | 200 | 10 | 65 | 70 |
| | (Technical) | | | | | |

Notes to above results table:
Assessed on a scale of 0-100% visual herbicidal damage, where 0 = no damage and 100 = total kill.
M = data point missing.
An empty box for % damage to Rice, ECHCG or LEFCH means that that particular mixture was not tested at the specified amounts either against rice or against the weeds. % damage to ECHCG and LEFCH was tested at 60 + 90 + 120 g/ha of compound A-4, A-7 and A-9. % damage to IR-64 rice was tested at 90 + 120 + 240 g/ha of compound A-4, A-7 and A-9.

Comments on the Results from Biological Example 1
1. Addition of fenoxasulfone to compound A-4 or A-7 increased the low (0 to 5%) herbicidal activity achieved by the compound A-4 (60 or 90 g/ha) or compound A-7 (60, 90 or 120 g/ha) alone against LEFCH.
2. Full (95%) control of LEFCH was achieved with 200 g/ha fenoxasulfone combined with any of: 60, 90 or 120 g/ha of compound A-4. 200 g/ha fenoxasulfone in mixture with as low as 60 g/ha of compound A-4 gave greater levels of control of LEFCH than 200 g/ha fenoxasulfone applied alone (which gave 70% LEFCH control).
3. 120 g/ha of compound A-7+200 g/ha fenoxasulfone gave full (90%) control of LEFCH. 60 or 90 g/ha of compound A-7+200 g/ha fenoxasulfone gave partial (70%) control of LEFCH.
3A. Compound A-9 alone showed high (80-90%) herbicidal activity against flooded LEFCH, with little (5%) damage (phytotoxicity) to the flooded transplanted IR-64 indica rice, when applied as the EC050 Formulation at an application rate of 60, 90 or 120 g/ha (note: no rice phytotoxicity measurement was performed at 60 g/ha).
4. Full (90-99%) control of ECHCG was achieved with 200 g/ha fenoxasulfone combined with any of: 60, 90 or 120 g/ha of compound A-4.
5. Control of ECHCG appeared to be numerically better with 200 g/ha fenoxasulfone combined with 60 or 90 g/ha of compound A-7 (65% and 70% control respectively), compared to the same (60 or 90 g/ha) rate of compound A-7 alone (35% and 55% control respectively).
6.120 g/ha of compound A-9 and 200 g/ha fenoxasulfone gave good control of both ECHCG (90%) and LEFCH (85%). 60 g/ha of compound A-9 and 200 g/ha fenoxasulfone gave good (90%) control of LEFCH.
7. Generally, the tested mixtures did not lessen damage (phytotoxicity) to the IR-64 variety rice (an indica rice) compared to the same application rate of compound A-4, A-7 or A-9 alone, except that 200 g/ha fenoxasulfone and 240 g/ha compound A-9 appeared to be numerically less phytotoxic on rice (5%) than 240 g/ha of compound A-9 alone (15% phytotoxicity on rice).
8. However, in some cases, inclusion of a mixture partner (fenoxasulfone or propyrisulfuron) with the compound A-4, A-7 or A-9 did improve herbicidal activity against ECHCG without a substantial increase in IR-64 rice damage, thus apparently improving the selectivity margin. These cases, with apparently improved IR-64 rice vs. ECHCG selectivity, were: 80 g/ha propyrisulfuron with compound A-4 (60 g/ha), A-7 (60+90 g/ha), or A-9 (60+90 g/ha); and also 200 g/ha fenoxasulfone with compound A-4 (60 g/ha) or A-9 (60+90+120 g/ha).
9. Mixtures of 200 g/ha fenoxasulfone with compound A-7 were substantially less phytotoxic to IR-64 rice than mixtures of 10 g/ha of the comparator co-herbicide pyroxasulfone with compound A-7.
10. Mixtures of 80 g/ha propyrisulfuron with compound A-4, A-7 or A-9 in general tended to be more efficacious against ECHCG than mixtures of 80 g/ha of the comparator co-herbicide imazosulfuron with compound A-4, A-7 or A-9.
11. 80 g/ha Propyrisulfuron+compound A-7 mixtures appear less phytotoxic to IR-64 rice than 80 g/ha imazosulfuron+compound A-7 mixtures.

### Biological Example 2

### Generalised Methods for Testing Mixtures of Compounds of Formula (I) and Co-Herbicides in or Direct Seeded Rice Systems or Transplanted Rice Systems

For the direct seeded system, monocotyledonous and/or dicotyledonous test weeds and/or rice plants (indica and/or japonica varieties) are sown in troughs at different intervals before the chemical application depending on the species and the desired growth stages required for the test (typically around 2 weeks). The plants so prepared are used to simulate a post-emergence application. Moreover, in order to simulate also a pre-emergence application, the same species plants are sown 1 or 2 days before the chemical applications. After application, the troughs are flooded at different intervals to simulate wet and dry seeded flooded rice systems.

The chemical application for direct seeded rice systems consists of spraying the plants with an aqueous spray solution derived from the formulation of the technical active ingredient in 0.6 ml acetone and 45 ml formulation solution containing 10.6% Emulsogen EL™ (castor oil ethoxylate, Registry number 61791-12-6), 42.2% N-methylpyrrolidone, 42.2% dipropylene glycol monomethyl ether (Registry number 34590-94-8) and 0.2% X-77 (Registry number 11097-66-8).

The plants included in the tests in direct seeded are Echinochloa crus-galli (ECHCG) at two growth stages (GS), 0 and 12-13 using BBCH scale, Leptochloa chinensis (LEFCH) at GS 12-13, Brachiaria spp at GS 12-13; Commelina spp at GS 12-13, Cyperus spp at GS 12-13 and Monochoria vaginalis (MOOVA) at GS 12-13 and other grasses; varieties of japonica and indica rice (for example, rice variety IR-64 (an indica rice) or Arborio) are included as well.

To simulate transplanted rice systems, monocotyledonous and dicotyledonous test weeds are sown following the same procedures as for the direct seeded rice system. The rice plants are initially grown in nursery trays until they reach the growth stage (GS) of 12-13 (using the BBCH scale), then they are transplanted in the same troughs alongside the previously sown weeds. After transplanting, the troughs are flooded. The chemical treatment is performed between 4 to 9 days after the transplantation (DAT) of rice. The chemical is applied as a 10 ml aliquot of chemical solution formulated as for the direct seeded system in each trough. The plants included in the tests in transplanted system are Echinochloa spp at two growth stages (0 and 12-13 BBCH scale); Cyperus spp at GS 12-13 and Monochoria vaginalis (MOOVA).

The test plants are grown in a greenhouse under optimum conditions for 21 days after application (DAA) of chemical. The test is evaluated at 14 and 21 DAA. The assessment consists of evaluating the visual damage of treated plants compared to an untreated control. The severity of damage is expressed using a 0-100 scale (0=no damage to plant; 100=plants were killed).

1. A herbicidal composition comprising as active ingredient a mixture of:
a) a herbicidally effective amount of a compound of formula (I) 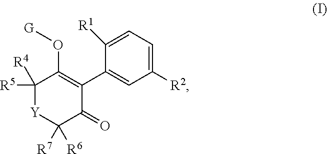
wherein:
R¹ is methyl, ethyl, n-propyl, halogen, difluoromethoxy, trifluoromethoxy or trifluoromethyl,
R² is phenyl or phenyl substituted by C₁-C₄ alkyl, C₁-C₄ haloalkyl, C₁-C₄ alkoxy, C₁-C₄ haloalkoxy or halogen,
R⁴, R⁵, R⁶ and R⁷, independently of each other, are hydrogen or C₁-C₄ alkyl,
Y is O, and
G is hydrogen, an alkali metal, alkaline earth metal, sulfonium, or ammonium, or G is a latentiating group which is C(O)—R^{a} or C(O)—O—R^{b};
wherein R^{a} is H, C₁-C₁₈alkyl, C₂-C₁₈alkenyl, C₂-C₁₈alkynyl, C₁-C₁₀haloalkyl, C₁-C₁₀cyanoalkyl, C₁-C₁₀nitroalkyl, C₁-C₁₀aminoalkyl, C₁-C₅alkylaminoC₁-C₅alkyl, C₂-C₈dialkylaminoC₁-C₅alkyl, C₃-C₇cycloalkylC₁-C₅alkyl, C₁-C₅alkoxyC₁-C₅alkyl, C₃-C₅alkenyloxyC₁-C₅alkyl, C₃-C₅alkynyloxyC₁-C₅alkyl, C₁-C₅alkylthioC₁-C₅alkyl, C₁-C₅alkylsulfinylC₁-C₅alkyl, C₁-C₅alkylsulfonylC₁-C₅alkyl, C₂-C₈alkylideneaminoxyC₁-C₅alkyl, C₁-C₅alkylcarbonylC₁-C₅alkyl, C₁-C₅alkoxycarbonylC₁-C₅alkyl, aminocarbonylC₁-C₅alkyl, C₁-C₅alkylaminocarbonylC₁-C₅alkyl, C₂-C₈dialkylaminocarbonylC₁-C₅alkyl, C₁-C₅alkylcarbonylaminoC₁-C₅alkyl, N—C₁-C₅alkylcarbonyl-N—C₁-C₅alkylaminoC₁-C₅alkyl, C₃-C₆-trialkylsilylC₁-C₅alkyl, phenylC₁-C₅alkyl (wherein the phenyl is optionally substituted by C₁-C₃alkyl, C₁-C₃haloalkyl, C₁-C₃alkoxy, C₁-C₃haloalkoxy, C₁-C₃alkylthio, C₁-C₃alkylsulfinyl, C₁-C₃alkylsulfonyl, halogen, cyano, or by nitro), heteroarylC₁-C₅alkyl (wherein the heteroaryl is optionally substituted by C₁-C₃alkyl, C₁-C₃haloalkyl, C₁-C₃alkoxy, C₁-C₃haloalkoxy, C₁-C₃alkylthio, C₁-C₃alkylsulfinyl, C₁-C₃alkylsulfonyl, halogen, cyano, or by nitro), C₂-C₅haloalkenyl, C₃-C₈cycloalkyl, phenyl or phenyl substituted by C₁-C₃alkyl, C₁-C₃haloalkyl, C₁-C₃alkoxy, C₁-C₃haloalkoxy, halogen, cyano or nitro, heteroaryl or heteroaryl substituted by C₁-C₃alkyl, C₁-C₃haloalkyl, C₁-C₃alkoxy, C₁-C₃haloalkoxy, halogen, cyano or nitro; and
R^{b} is C₁-C₁₈alkyl, C₃-C₁₈alkenyl, C₃-C₁₈alkynyl, C₂-C₁₀haloalkyl, C₁-C₁₀cyanoalkyl, C₁-C₁₀nitroalkyl, C₂-C₁₀aminoalkyl, C₁-C₅alkylaminoC₁-C₅alkyl, C₂-C₈dialkylaminoC₁-C₅alkyl, C₃-C₇cycloalkylC₁-C₅alkyl, C₁-C₅alkoxyC₁-C₅alkyl, C₃-C₅alkenyloxyC₁-C₅alkyl, C₃-C₅alkynyloxyC₁-C₅alkyl, C₁-C₅alkylthioC₁-C₅alkyl, C₁-C₅alkylsulfinylC₁-C₅alkyl, C₁-C₅alkylsulfonylC₁-C₅alkyl, C₂-C₈alkylideneaminoxyC₁-C₅alkyl, C₁-C₅alkylcarbonylC₁-C₅alkyl, C₁-C₅alkoxycarbonylC₁-C₅alkyl, aminocarbonylC₁-C₅alkyl, C₁-C₅alkylaminocarbonylC₁-C₅alkyl, C₂-C₈dialkylaminocarbonylC₁-C₅alkyl, C₁-C₅alkylcarbonylaminoC₁-C₅alkyl, N—C₁-C₅alkylcarbonyl-N—C₁-C₅alkylaminoC₁-C₅alkyl, C₃-C₆-trialkylsilylC₁-C₅alkyl, phenylC₁-C₅alkyl (wherein the phenyl is optionally substituted by C₁-C₃alkyl, C₁-C₃haloalkyl, C₁-C₃alkoxy, C₁-C₃haloalkoxy, C₁-C₃alkylthio, C₁-C₃alkylsulfinyl, C₁-C₃alkylsulfonyl, halogen, cyano, or by nitro), heteroarylC₁-C₅alkyl (wherein the heteroaryl is optionally substituted by C₁-C₃alkyl, C₁-C₃haloalkyl, C₁-C₃alkoxy, C₁-C₃haloalkoxy, C₁-C₃alkylthio, C₁-C₃alkylsulfinyl, C₁-C₃alkylsulfonyl, halogen, cyano, or by nitro), C₃-C₅haloalkenyl, C₃-C₈cycloalkyl, phenyl or phenyl substituted by C₁-C₃alkyl, C₁-C₃haloalkyl, C₁-C₃alkoxy, C₁-C₃haloalkoxy, halogen, cyano or nitro, heteroaryl or heteroaryl substituted by C₁-C₃alkyl, C₁-C₃haloalkyl, C₁-C₃alkoxy, C₁-C₃haloalkoxy, halogen, cyano or nitro;
and
b) a co-herbicide selected from the group consisting of fenoxasulfone, ipfencarbazone, propyrisulfuron, and N-[2-[(4,6-dimethoxy-1,3,5-triazin-2-yl)carbonyl]-6-fluorophenyl]-1,1-difluoro-N-methylmethanesulfonamide.

2. A herbicidal composition as claimed in claim 1, wherein R¹ is ethyl.

3. A herbicidal composition as claimed in claim 1, wherein R² is phenyl substituted by methyl, methoxy, or halogen.

4. A herbicidal composition as claimed in claim 3, wherein R² is phenyl substituted by fluorine or chlorine.

5. A herbicidal composition as claimed in claim 1, wherein R⁴, R⁵, R⁶ and R⁷, independently of each other, are hydrogen or C₁-C₂ alkyl.

6. A herbicidal composition as claimed in claim 5, wherein R⁴, R⁵, R⁶ and R⁷ are methyl.

7. A herbicidal composition as claimed in claim 1, wherein G is hydrogen, C(O)—R^{a} or C(O)—O—R^{b}; wherein R^{a} and R^{b} are C₁-C₆alkyl.

8. A herbicidal composition as claimed in claim 7, wherein G is hydrogen, C(O)—R^{a} or C(O)—O—R^{b}; wherein R^{a} and R^{b} are methyl, ethyl, n-propyl, isopropyl or t-butyl.

9. A herbicidal composition as claimed in claim 8, wherein G is hydrogen.

10. A herbicidal composition as claimed in claim 1, wherein the compound of formula (I) is:

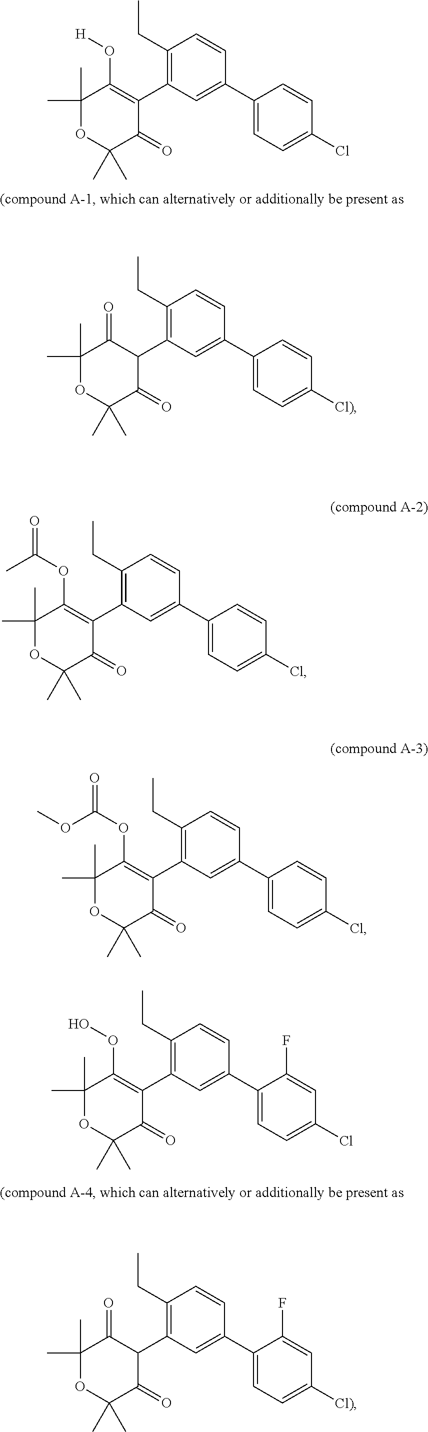

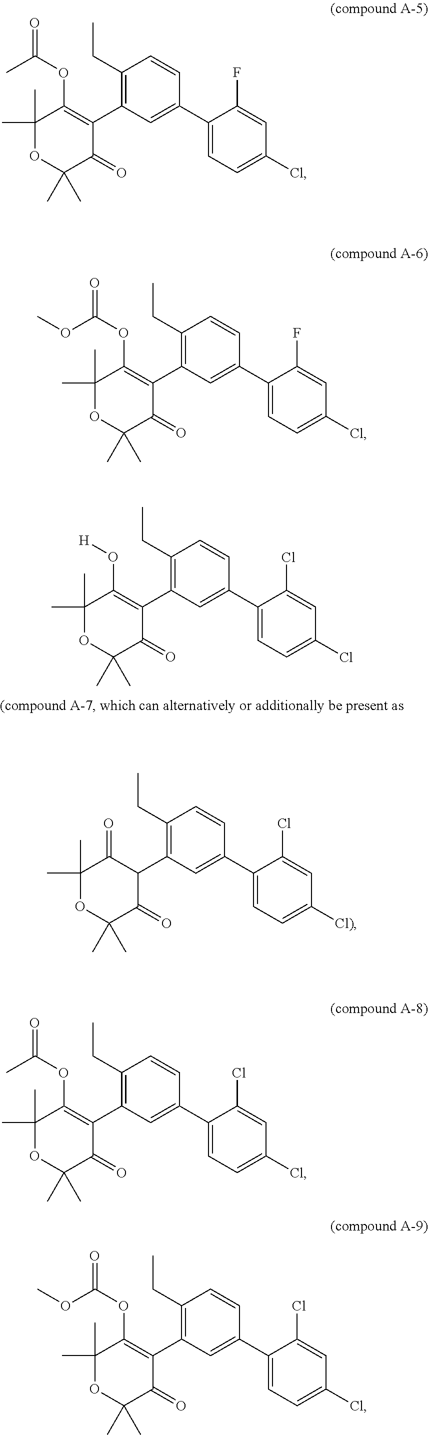

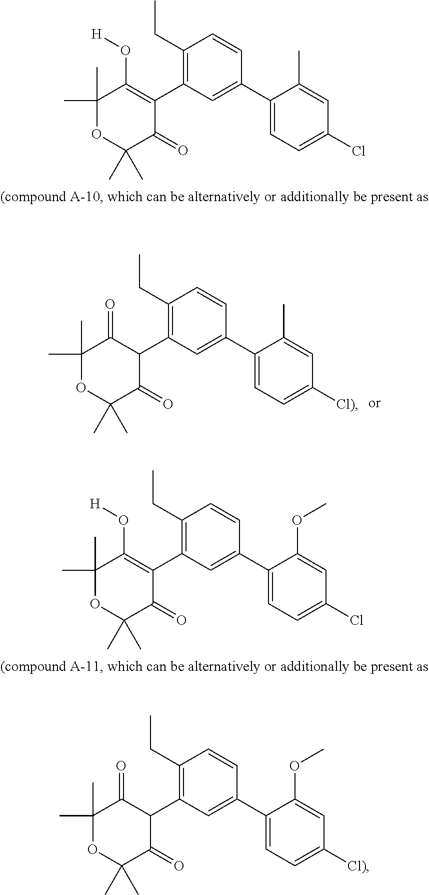

11. A herbicidal composition as claimed in claim 10, wherein the compound of formula (I) is compound A-4, A-7 or A-9.

12. A herbicidal composition as claimed in claim 1, wherein the co-herbicide is fenoxasulfone or ipfencarbazone.

13. A herbicidal composition as claimed in claim 1, wherein the co-herbicide is fenoxasulfone.

14. A herbicidal composition as claimed in claim 1, wherein the co-herbicide is ipfencarbazone.

15. A herbicidal composition as claimed in claim 1, wherein the co-herbicide is propyrisulfuron.

16. A herbicidal composition as claimed in claim 1, wherein the co-herbicide is N-[2-[(4,6-dimethoxy-1,3,5-triazin-2-yl)carbonyl]-6-fluorophenyl]-1,1-difluoro-N-methylmethanesulfonamide.

17. A herbicidal composition as claimed in claim 11, wherein the co-herbicide is fenoxasulfone or ipfencarbazone.

18. A herbicidal composition as claimed in claim 11, wherein the co-herbicide is fenoxasulfone.

19. A herbicidal composition as claimed in claim 18, wherein the compound of formula (I) is compound A-4, and the co-herbicide is fenoxasulfone.

20. A herbicidal composition as claimed in claim 18, wherein the compound of formula (I) is compound A-7, and the co-herbicide is fenoxasulfone.

21. A herbicidal composition as claimed in claim 18, wherein the compound of formula (I) is compound A-9, and the co-herbicide is fenoxasulfone.

22. A herbicidal composition as claimed in claim 1, wherein the weight ratio of the compound of formula (I) to the fenoxasulfone is from 1:6 to 3:2.

23. A herbicidal composition as claimed in any claim 22, wherein the weight ratio of the compound of formula (I) to the fenoxasulfone is from 1:4 to 4:5.

24. A herbicidal composition as claimed in claim 23, wherein the weight ratio of the compound of formula (I) to the fenoxasulfone is from 3:10 to 7:10.

25. A herbicidal composition as claimed in claim 24, wherein the weight ratio of the compound of formula (I) to the fenoxasulfone is from 2:5 to 7:10.

26. A herbicidal composition as claimed in claim 25, wherein the weight ratio of the compound of formula (I) to the fenoxasulfone is from 1:2 to 7:10.

27. A herbicidal composition as claimed in claim 11, wherein the co-herbicide is ipfencarbazone.

28. A herbicidal composition as claimed in claim 1, wherein the weight ratio of the compound of formula (I) to the ipfencarbazone is from 1:7 to 1:1.

29. A herbicidal composition as claimed in claim 28, wherein the weight ratio of the compound of formula (I) to the ipfencarbazone is from 6:25 to 1:2.

30. A herbicidal composition as claimed in claim 11, wherein the co-herbicide is propyrisulfuron.

31. A herbicidal composition as claimed in claim 1, wherein the weight ratio of the compound of formula (I) to the propyrisulfuron is from 1:2 to 3:1.

32. A herbicidal composition as claimed in claim 31, wherein the weight ratio of the compound of formula (I) to the propyrisulfuron is from 3:4 to 3:2.

33. A herbicidal composition as claimed in claim 11, wherein the co-herbicide is N-[2-[(4,6-dimethoxy-1,3,5-triazin-2-yl)carbonyl]-6-fluorophenyl]-1,1-difluoro-N-methylmethanesulfonamide.

34. A herbicidal composition as claimed in claim 1, wherein the weight ratio of the compound of formula (I) to the N-[2-[(4,6-dimethoxy-1,3,5-triazin-2-yl)carbonyl]-6-fluorophenyl]-1,1-difluoro-N-methylmethanesulfonamide is from 1:20 to 20:1.

35. A herbicidal composition as claimed in claim 1, which is a formulation comprising a carrier, a solvent and/or a surface-active substance.

36. A herbicidal composition as claimed in claim 35, wherein the formulation is in the form of a wettable powder, a water-dispersible granule, an emulsifiable concentrate, a microemulsifiable concentrate, an oil-in-water emulsion, an oil flowable, an aqueous dispersion, an oily dispersion, a soluble liquid, or a water-soluble concentrate wherein the water-soluble concentrate is with water or a water-miscible organic solvent as carrier.

37. A herbicidal composition as claimed in claim 36, wherein the formulation is in the form of an emulsifiable concentrate.

38. A herbicidal composition as claimed in claim 1, comprising c) a safener and, optionally, d) an oil additive.

39. A herbicidal composition as claimed in to claim 38 comprising c) a safener selected from cloquintocet-mexyl and mefenpyr-diethyl.

40. A herbicidal composition as claimed in claim 1, which is for controlling grasses and weeds in crops of useful plants.

41. A herbicidal composition as claimed in claim 40, which is for controlling grasses and weeds in crops of rice.

42. A method of controlling grasses and weeds in crops of useful plants, which comprises applying a herbicidal composition as defined in claim 1 to the plants or to the locus thereof.

43. A method as claimed in claim 42, wherein the crops of useful plants are crops of rice.

44. A method as claimed in claim 43, wherein the crops of useful plants are crops of flooded transplanted rice.

45. A method as claimed in claim 42, wherein the grasses and weeds controlled comprise Echinochloa and/or Leptochloa.

46. A method as claimed in claim 42, wherein the herbicidal composition is applied to the plants or to the locus thereof at an application rate of 30 to 240 g of the compound of formula (I) per hectare, calculated as the weight of the compound of formula (I) excluding the weight of any optional counterions thereof.

47. A method as claimed in claim 46, wherein the herbicidal composition is applied to the plants or to the locus thereof at an application rate of 50 to 150 g of the compound of formula (I) per hectare, calculated as the weight of the compound of formula (I) excluding the weight of any optional counterions thereof.

48. A method as claimed in claim 42, wherein the co-herbicide is fenoxasulfone, ipfencarbazone, or propyrisulfuron; and
when the co-herbicide is fenoxasulfone, then the herbicidal composition is applied to the plants or to the locus thereof at an application rate of 100 to 400 g of fenoxasulfone per hectare, calculated as the weight of fenoxasulfone excluding the weight of any optional counterions thereof; and
when the co-herbicide is ipfencarbazone, then the herbicidal composition is applied to the plants or to the locus thereof at an application rate of 100 to 500 g of ipfencarbazone per hectare, calculated as the weight of ipfencarbazone excluding the weight of any optional counterions thereof; and
when the co-herbicide is propyrisulfuron, then the herbicidal composition is applied to the plants or to the locus thereof at an application rate of 40 to 160 g of propyrisulfuron per hectare, calculated as the weight of propyrisulfuron excluding the weight of any optional counterions thereof.

49. A method as claimed in claim 48, wherein the co-herbicide is fenoxasulfone, ipfencarbazone, or propyrisulfuron; and
when the co-herbicide is fenoxasulfone, then the herbicidal composition is applied to the plants or to the locus thereof at an application rate of 200 g of fenoxasulfone per hectare, calculated as the weight of fenoxasulfone excluding the weight of any optional counterions thereof; and
when the co-herbicide is ipfencarbazone, then the herbicidal composition is applied to the plants or to the locus thereof at an application rate of 250 g of ipfencarbazone per hectare, calculated as the weight of ipfencarbazone excluding the weight of any optional counterions thereof; and
when the co-herbicide is propyrisulfuron, then the herbicidal composition is applied to the plants or to the locus thereof at an application rate of 80 g of propyrisulfuron per hectare, calculated as the weight of propyrisulfuron excluding the weight of any optional counterions thereof.

50. A method of controlling Leptochloa weeds in crops of useful plants, which comprises applying compound A-9, whose structure is 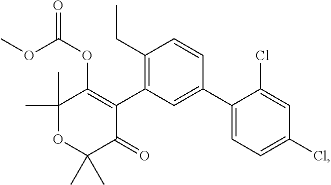 [SMILES: CCc1ccc(-c2ccc(Cl)cc2Cl)cc1C1=C(OC(=O)OC)C(C)(C)OC(C)(C)C1=O]
to the plants or to the locus thereof.
